(19) United States
(12) Patent Application Publication
(10) Pub. No.: US 2010/0056570 A1
(43) Pub. Date: Mar. 4, 2010

(54) FUNGICIDES

(75) Inventors: Kessabi Fiona Murphy, Basel (CH); Renaund Beaudeginies, Basel (CH); Laura Quaranta, Basel (CH); Hans-Georg Brunner, Basel (CH); Fredrik Cederbaum, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: Syngenta Crop Protection, Inc., Greensboro, NC (US)

(21) Appl. No.: 12/439,834

(22) PCT Filed: Sep. 4, 2007
(86) PCT No.: PCT/EP2007/007700

§ 371 Date: Mar. 4, 2009

(30) Foreign Application Priority Data
Sep. 6, 2006 (US) 0617574.9

(51) Int. Cl. A01N 43/42; C07D 215/16; A01P 3/00

(52) U.S. Cl. 514/314; 546/178; 514/311; 546/167

(57) ABSTRACT

Compounds of the general formula wherein the substituents are as defined in claim (1), are useful as fungicides.

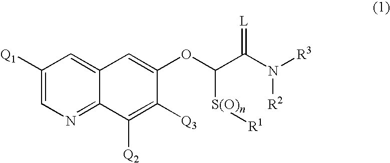

This invention relates to novel quinolinyloxyalkanoic acid amides, processes for preparing them, to compositions containing them and to methods of using them to combat fungi, especially fungal infections of plants.

Certain quinolinyloxyalkanoic acid amide derivatives and their use as agricultural and horticultural bactericides are disclosed, for example, in WO 04/047538 and JP 2001-89453.

The present invention is concerned with the provision of particular substituted quinoline-6-yloxyalkanoic acid amides for use mainly as plant fungicides.

Thus according to the present invention there is provided a compound of the general formula I

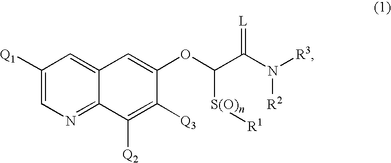

wherein
Q¹, Q² and Q³, independently of each other, are halogen, cyano, nitro, azido, optionally substituted C₁₋₆ alkyl, optionally substituted C₃₋₆ cycloalkyl, optionally substituted C₃₋₆-heterocyclyl, which contains at least one heteroatom selected from sulphur, oxygen or NR⁰, where R⁰ is hydrogen or optionally substituted C₁₋₆alkyl, optionally substituted C₃₋₆ cycloalkyl(C₁₋₄)alkyl, optionally substituted C₂₋₆ alkenyl, optionally substituted C₂₋₆ alkynyl, optionally substituted C₁₋₆ alkoxy, optionally substituted C₂₋₆ alkenyloxy, optionally substituted C₂₋₆ alkynyloxy, optionally substituted aryl, optionally substituted aryloxy, optionally substituted aryl(C₁₋₆)alkyl, optionally substituted aryl(C₁₋₆)alkoxy, optionally substituted heteroaryl, optionally substituted heteroaryloxy, optionally substituted heteroaryl(C₁₋₆)alkyl, optionally substituted heteroaryl(C₁₋₆)alkoxy, —SF₅ or —S(O)ᵤ(C₁₋₆)alkyl, wherein u is 0, 1 or 2 and the alkyl group is optionally substituted with halogen, or
Q¹, Q² and Q³, independently of each other, are —OSO₂(C₁₋₄)alkyl, wherein the alkyl group is optionally substituted with halogen, or
Q¹, Q² and Q³, independently of each other, are —CONR^{u}R^{v}, —COR^{u}, —CO₂R^{u}, —CR^{u}═NR^{v}, —NR^{u}R^{v}, —NR^{u}COR^{v}, —NR^{u}CO₂R^{v}, —SO₂NR^{u}R^{v} or —NR^{u}SO₂R^{w}, wherein R^{w} is optionally substituted C₁₋₆ alkyl and R^{u} and R^{v}, independently of each other, are hydrogen or C₁₋₆ alkyl optionally substituted with halogen, or, in the case of —CONR^{u}R^{v} or —SO₂NR^{u}R^{v}, R^{u}R^{v} may join to form a 5- or 6-membered carbocyclic or heterocyclic ring containing a heteroatom selected from sulfur, oxygen and NR^{o}, wherein R^{o} is hydrogen or optionally substituted C₁₋₆alkyl, or, in the case of —CR^{u}═NR^{v}, R^{v} is hydroxy or optionally substituted C₁₋₆alkoxy, optionally substituted aryloxy or optionally substituted heteroaryloxy, or
Q¹ and Q², independently of each other, additionally denote hydrogen,
R¹ is optionally substituted C₁₋₄ alkyl, optionally substituted C₂₋₄ alkenyl, optionally substituted C₂₋₄ alkynyl or optionally substituted C₃₋₄ cycloalkyl,
R² is hydrogen, C₁₋₈ alkyl, C₃₋₄ cycloalkyl, C₂₋₈ alkenyl, cyano, hydroxy, alkoxy, cyano(C₁₋₄)alkyl, C₁₋₄ alkoxy(C₁₋₄)alkyl, C₁₋₄ alkoxy(C₁₋₄)alkoxy(C₁₋₄)alkyl or benzyloxy(C₁₋₄)alkyl, wherein the phenyl ring is optionally substituted with C₁₋₄ alkoxy,
R³ is (CR^{a}R^{b})ₚ(CR^{c}R^{d})_{q}(X)ᵣ(CR^{e}R^{f})ₛR⁴, wherein
R^{a}, R^{b}, R^{c}, R^{d}, R^{e} and R^{f}, independently of each other, are hydrogen, C₁₋₄ alkyl, halogen, cyano, hydroxy, C₁₋₄ alkoxy or C₁₋₄ alkoxycarbonyl, or
R^{a}R^{b}, R^{c}R^{d} or R^{e}R^{f} may join to form a 3 to 8 membered carbocyclic or heterocyclic ring containing a heteroatom selected from sulfur, oxygen and NR^{o}, wherein R^{o} is hydrogen or optionally substituted C₁₋₆alkyl,
X is (CO), (CO)O, O(CO), O, S(O)ₜ, wherein t is 0, 1 or 2, or X is NH or N(C₁₋₆)alkyl,
p, r and s, independently of each other, are 0 or 1,
q is 0, 1 or 2,
R⁴ is optionally substituted C₁₋₆ alkyl, optionally substituted C₂₋₆ alkenyl, —CR^{uu}═NR^{vv}, wherein R^{uu} is hydrogen or C₁₋₆ alkyl and R^{vv} is hydroxy or optionally substituted C₁₋₆alkoxy, optionally substituted aryloxy or optionally substituted heteroaryloxy, or —CH₂—C≡C—R⁵, wherein
R⁵ is hydrogen, C₁₋₈ alkyl optionally substituted with halogen, hydroxy, C₁₋₆ alkoxy, C₁₋₃ alkoxy(C₁₋₃)alkoxy, cyano, C₁₋₄ alkylcarbonyloxy, aminocarbonyloxy, mono- or di(C₁₋₄)-alkylaminocarbonyloxy, tri(C₁₋₄)alkylsilyloxy or —S(O)_{g}(C₁₋₆)alkyl, wherein g is 0, 1 or 2, or R⁵ is C₃₋₆ cycloalkyl optionally substituted with halogen, hydroxy, C₁₋₆ alkoxy, C₁₋₃ alkoxy-(C₁₋₃)alkoxy, cyano, C₁₋₄ alkylcarbonyloxy, aminocarbonyloxy, mono- or di(C₁₋₄)alkyl-aminocarbonyloxy, tri(C₁₋₄)alkylsilyloxy or —S(O)_{g}(C₁₋₆)alkyl, wherein g is 0, 1 or 2, or R⁵ is C₃₋₆ cycloalkyl(C₁₋₄)alkyl, wherein the alkyl and/or cycloalkyl moiety is optionally substituted with halogen, hydroxy, C₁₋₆ alkoxy, C₁₋₃ alkoxy(C₁₋₃)alkoxy, cyano, C₁₋₄ alkyl-carbonyloxy, aminocarbonyloxy, mono- or di(C₁₋₄)alkylaminocarbonyloxy, tri(C₁₋₄)alkyl-silyloxy or —S(O)_{g}(C₁₋₆)alkyl, wherein g is 0, 1 or 2, or
R⁵ is optionally substituted aryl, optionally substituted aryl(C₁₋₄)alkyl, optionally substituted aryloxy(C₁₋₄)alkyl, optionally substituted heteroaryl or optionally substituted heteroaryl(C₁₋₄)alkyl or optionally substituted heteroaryloxy(C₁₋₄)alkyl, or
R⁴ is optionally substituted C₃₋₆ cycloalkyl, optionally substituted C₅₋₆ cycloalkenyl, optionally substituted aryl, optionally substituted heteroaryl or an optionally substituted 5- to 8-membered ring optionally containing a heteroatom selected from sulfur, oxygen or NR⁰, wherein R^{o} is hydrogen or optionally substituted C₁₋₆alkyl, or
when R³ is (CR^{a}R^{b})ₚ(CR^{c}R^{d})_{q}(X)ᵣ(CR^{e}R^{f})ₛR⁴R² and R³ may join to form a 5- or 6-membered ring optionally substituted with halogen, C₁₋₄ alkyl, mono- or di-(C₁₋₄)alkylaminocarbonyl, and optionally containing a heteroatom selected from sulphur, oxygen and NR⁰⁰, wherein R⁰⁰ is C₁₋₄ alkyl optionally substituted with halogen, C₁₋₆ alkoxy or cyano, or R⁰⁰ is phenyl optionally substituted with nitro, C₁₋₄ alkyl, halo(C₁₋₄)-alkyl, C₁₋₄ alkylcarbonyl or heteroaryl, or R² and R³ may join to form an optionally substituted 6,6-membered bicycle,
R³ is —(CR³⁰R⁴⁰)C≡CR⁵⁰, wherein
R³⁰ and R⁴⁰, independently of each other, are hydrogen, C₁₋₆alkyl, halo(C₁₋₄)alkyl, C₁₋₄ alkoxy(C₁₋₃)alkyl, C₂₋₃ alkenyl or C₂₋₃alkynyl, or
R³⁰ and R⁴⁰ join with the carbon atom to which they are attached to form a 3 to 6 membered carbocyclic or heterocyclic ring containing a heteroatom selected from sulfur, oxygen or NR⁰⁰⁰, wherein R⁰⁰⁰ is hydrogen or C₁₋₄ alkyl, where the carbocyclic or heterocyclic ring is optionally substituted with halo or C₁₋₄alkyl,
R⁵⁰ is hydrogen, optionally substituted C₁₋₄ alkyl, optionally substituted C₃₋₆ cycloalkyl, optionally substituted aryl or optionally substituted heteroaryl containing a heteroatom selected from sulphur, oxygen or NR⁰⁰⁰, wherein R⁰⁰⁰ is hydrogen or C₁₋₆alkyl,
L is sulfur or oxygen, and
n is 0, 1 or 2, and
salts and N-oxides of the compounds of the formula I.

The compounds of the invention contain at least one asymmetric carbon atom and may exist as enantiomers (or as pairs of diastereoisomers) or as mixtures of such. Further, when n is 1, the compounds of the invention are sulphoxides, which can exists in two enantiomeric forms, and the adjacent carbon can also exists in two enantiomeric forms. Compounds of general formula (I) can therefore exist as racemates, diastereoisomers, or single enantiomers, and the invention includes all possible isomers or isomer mixtures in all proportions. It is to be expected that for any given compound, one isomer may be more fungicidally active than another.

N-oxides of the compounds of the formula I preferably denote the N-oxides formed by the quinoline moiety.

The salts which the compounds of the formula I can form are preferably those formed by interaction of these compounds with acids. The term “acid” comprises mineral acids such as hydrogen halides, sulphuric acid, phosphoric acid etc. as well as organic acids, preferably the commonly used alkanoic acids, for example formic acid, acetic acid and propionic acid.

Except where otherwise stated, alkyl groups and alkyl moieties of alkoxy, alkylthio, etc., suitably contain from 1 to 6, typically from 1 to 4, carbon atoms in the form of straight or branched chains. Examples are methyl, ethyl, n- and iso-propyl and n-, sec-, iso- and tert-butyl. Where alkyl moieties contain 5 or 6 carbon atoms, examples are n-pentyl and n-hexyl. Examples of suitable optional substituents of alkyl groups and moieties include halo, hydroxy, C₁₋₄ alkoxy and C₁₋₄ alkoxy(C₁₋₄)alkoxy, cyano, optionally substituted aryl and optionally substituted heteroaryl. Where the optional substituent is halo, the haloalkyl group or moiety is typically monochloromethyl, monofluoromethyl, dichloromethyl, difluoromethyl, trichloromethyl or trifluoromethyl.

Except where otherwise stated, alkenyl and alkynyl moieties also suitably contain from 2 to 6, typically from 2 to 4, carbon atoms in the form of straight or branched chains. Examples are allyl, ethynyl and propargyl. Optional substituents include halo, alkoxy, optionally substituted aryl and optionally substituted heteroaryl.

Halo includes fluoro, chloro, bromo and iodo.

Aryl is usually phenyl but also includes naphthyl, anthryl and phenanthryl.

Heteroaryl is typically a 5- or 6-membered aromatic ring containing one or more sulphur, oxygen or NR moieties as heteroatoms, which may be fused to one or more other aromatic or heteroaromatic rings, such as a benzene ring. Examples are thienyl, furyl, pyrrolyl, isoxazolyl, oxazolyl, thiazolyl, oxadiazolyl, pyrazolyl, imidazolyl, triazolyl, isothiazolyl, tetrazolyl, thiadiazolyl, pyridyl, pyrimidinyl, pyrazinyl, pyridazinyl, triazinyl, benzofuranyl, benzothienyl, dibenzofuranyl, dibenzothienyl, benzothiazolyl, benzoxazolyl, benzimidazolyl, indolyl, quinolyl, isoquinolyl, quinazolinyl and quinoxalinyl groups and, where appropriate, N-oxides and salts thereof. Any of the aryl or heteroaryl values are optionally substituted. Except where otherwise stated, substituents which may be present include one or more of the following: halo, hydroxy, mercapto, C₁₋₆ alkyl (especially methyl and ethyl), C₂₋₆ alkenyl (especially allyl), C₂₋₆ alkynyl (especially propargyl), C₁₋₆ alkoxy (especially methoxy), C₂₋₆ alkenyloxy (especially allyloxy), C₂₋₆ alkynyloxy (especially propargyloxy), halo(C₁₋₆)alkyl (especially trifluoromethyl), halo(C₁₋₆)alkoxy (especially trifluoromethoxy), —S(O)ₘ(C₁₋₆)alkyl wherein m is 0, 1 or 2 and the alkyl is optionally substituted with halo, hydroxy(C₁₋₆)alkyl, C₁₋₄alkoxy(C₁₋₄)alkyl, C₁₋₄alkoxy(C₁₋₄)alkoxy, C₃₋₆ cycloalkyl, C₃₋₆ cycloalkyl(C₁₋₄)alkyl, optionally substituted aryl (especially optionally substituted phenyl), optionally substituted heteroaryl (especially optionally substituted pyridyl or pyrimidinyl), optionally substituted aryloxy (especially optionally substituted phenoxy), optionally substituted heteroaryloxy (especially optionally substituted pyridyloxy or pyrimidinyloxy), optionally substituted —S(O)ₘaryl wherein m is 0, 1 or 2 (especially optionally substituted phenylthio), optionally substituted —S(O)ₘheteroaryl wherein m is 0, 1 or 2 (especially optionally substituted pyridylthio or pyrimidinylthio), optionally substituted aryl(C₁₋₄)alkyl (especially optionally substituted benzyl, optionally substituted phenethyl and optionally substituted phenyl n-propyl) in which the alkyl moiety is optionally substituted with hydroxy, optionally substituted heteroaryl(C₁₋₄)alkyl (especially optionally substituted pyridyl- or pyrimidinyl-(C₁₋₄)alkyl), optionally substituted aryl(C₂₋₄)alkenyl (especially optionally substituted phenylethenyl), optionally substituted heteroaryl(C₂₋₄)alkenyl (especially optionally substituted pyridylethenyl or pyrimidinylethenyl), optionally substituted aryl(C₁₋₄)alkoxy (especially optionally substituted benzyloxy and phenethyloxy), optionally substituted heteroaryl(C₁₋₄)alkoxy (especially optionally substituted pyridyl(C₁₋₄)alkoxy or pyrimidinyl(C₁₋₄)alkoxy), optionally substituted aryloxy(C₁₋₄)alkyl (especially phenoxymethyl), optionally substituted heteroaryloxy-(C₁₋₄)alkyl (especially optionally substituted pyridyloxy or pyrimidinyloxy(C₁₋₄)alkyl), optionally substituted —S(O)ₘ(C₁₋₄)alkylaryl wherein m is 0, 1 or 2 (especially optionally substituted benzylthio and phenethylthio), optionally substituted —S(O)ₘ(C₁₋₄)alkylheteroaryl wherein m is 0, 1 or 2 (especially optionally substituted pyridyl(C₁₋₄)alkylthio or pyrimidinyl(C₁₋₄)alkylthio), optionally substituted —(C₁₋₄)alkylS(O)ₘaryl wherein m is 0, 1 or 2 (especially phenylthiomethyl), optionally substituted —(C₁₋₄)alkyl S(O)ₘheteroaryl wherein m is 0, 1 or 2 (especially optionally substituted pyridylthio(C₁₋₄)alkyl or pyrimidinylthio(C₁₋₄)alkyl), acyloxy, including C₁₋₄ alkanoyloxy (especially acetyloxy) and benzoyloxy, cyano, isocyano, thiocyanato, isothiocyanato, nitro, NR^{g}R^{h}, —NHCOR^{g}, —NHCONR^{g}R^{h}, —CONR^{g}R^{h}, —CO₂R^{g}, —SO₂Rⁱ, —OSO₂Rⁱ, —COR^{g}, —CR^{g}═NR^{h} or —N═CR^{g}R^{h} in which Rⁱ is C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄ alkyl or C₁₋₄ alkoxy and R^{g} and R^{h} are independently hydrogen, C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cyclo-alkyl(C₁₋₄)alkyl, phenyl or benzyl, the phenyl and benzyl groups being optionally substituted with halogen, C₁₋₄ alkyl or C₁₋₄ alkoxy.

Of particular interest are those compounds of the formula (I), wherein Q² is hydrogen, Q¹ and Q³ are as above.

Another group of preferred compounds of the formula (I) are those, wherein Q¹ is halogen, aryl or heteroaryl, Q² is hydrogen and Q³ is as above.

Another group of preferred compounds of the formula (I) are those, wherein Q¹ is aryl, Q² is hydrogen and Q³ is as defined above.

Another group of preferred compounds of the formula (I) are those, wherein Q¹ is heteroaryl, Q² is hydrogen and Q³ is as defined above.

Another group of preferred compounds of the formula (I) are those, wherein Q¹ and Q³ are halogen and Q² is hydrogen.

Another group of preferred compounds of the formula (I) are those, wherein, Q¹ is aryl or heteroaryl, Q² is hydrogen and Q³ is halogen.

Another group of preferred compounds of the formula (I) are those, wherein Q¹ and Q² are hydrogen and Q³ is halogen or optionally substituted alkyl.

Another group of preferred compounds of the formula (I) are those, wherein Q¹ is halogen, Q² is hydrogen and Q³ is optionally substituted alkyl.

Another group of preferred compounds of the formula (I) are those, wherein Q¹ and Q² are halogen and Q³ is optionally substituted alkyl.

Another group of preferred compounds of the formula (I) are those, wherein Q¹ is bromo.

Another group of preferred compounds of the formula (I) are those, wherein Q¹ is iodo.

Another group of preferred compounds of the formula (I) are those, wherein Q¹ is chloro.

Another group of preferred compounds of the formula (I) are those, wherein Q¹ is fluoro.

Another group of preferred compounds of the formula (I) are those, wherein Q³ is halogen.

Another group of preferred compounds of the formula (I) are those, wherein Q³ is fluorine.

Another group of preferred compounds of the formula (I) are those, wherein Q¹ is bromo, Q² is hydrogen and Q³ is fluoro.

Another group of preferred compounds of the formula (I) are those, wherein Q¹ is bromo, Q² is hydrogen and Q³ is chloro.

Another group of preferred compounds of the formula (I) are those, wherein Q¹ is iodo, Q² is hydrogen and Q³ is fluoro.

Another group of preferred compounds of the formula (I) are those, wherein Q¹ is iodo, Q² is hydrogen and Q³ is chloro.

Another group of preferred compounds of the formula (I) are those, wherein Q¹ is hydrogen, halogen, aryl or heteroaryl.

Another group of preferred compounds of the formula (I) are those, wherein R¹ is C₁₋₄ alkyl or halo(C₁₋₄)alkyl.

Another group of preferred compounds of the formula (I) are those, wherein R¹ is methyl.

Another group of preferred compounds of the formula (I) are those, wherein R¹ is ethyl.

Another group of preferred compounds of the formula (I) are those, wherein R² is hydrogen or methyl.

Another group of preferred compounds of the formula (I) are those, wherein R² is hydrogen.

Another group of preferred compounds of the formula (I) are those, wherein Q¹, Q² and Q³ are halogen.

Another group of preferred compounds of the formula (I) are those, wherein Q¹ is halogen, Q² is C₁₋₄alkyl and Q³ is halogen.

Another group of preferred compounds of the formula (I) are those, wherein Q¹ is halogen, Q² is C₁₋₄alkyl and Q³ is chloro.

Another group of preferred compounds of the formula (I) are those, wherein Q¹ is halogen, Q² is C₁₋₄alkyl and Q³ is fluoro.

Another group of preferred compounds of the formula (I) are those, wherein Q¹ is halogen, Q² is methyl and Q³ is halogen.

Another group of preferred compounds of the formula (I) are those, wherein Q¹ is halogen, Q² is methyl and Q³ is chloro.

Another group of preferred compounds of the formula (I) are those, wherein Q¹ is halogen, Q² is methyl and Q³ is fluoro.

Another group of preferred compounds of the formula (I) are those, wherein Q¹ is bromo, Q² is methyl and Q³ is chloro.

Another group of preferred compounds of the formula (I) are those, wherein Q¹ is bromo, Q² is methyl and Q³ is fluoro.

Another group of preferred compounds of the formula (I) are those, wherein Q¹ is iodo, Q² is methyl and Q³ is chloro.

Another group of preferred compounds of the formula (I) are those, wherein Q¹ is iodo, Q² is methyl and Q³ is fluoro.

Another group of preferred compounds of the formula (I) are those, wherein Q¹ is halogen, Q² and Q³ are C₁₋₄alkyl.

Another group of preferred compounds of the formula (I) are those, wherein R³ is tert-butyl, 1-halo-2-methylprop-2-yl, 1,1-dihalo-2-methylprop-2-yl, 1,1,1-trihalo-2-methylprop-2-yl, 1-alkoxy-2-methylprop-2-yl, 1-alkenyloxy-2-methylprop-2-yl, 1-alkynyloxy-2-methylprop-2-yl, 1-cyano-2-methyl-prop-2-yl, 1-alkoxyalkoxy-2-methyl-prop-2-yl, 1-halo-3-methylbut-3-yl, 1,1-dihalo-3-methylbut-3-yl, 1,1,1-trihalo-3-methylbut-3-yl, 1-alkoxy-3-methylbut-3-yl, 1-alkenyloxy-3-methylbut-3-yl, 1-alkynyloxy-3-methylbut-3-yl, 1-cyano-3-methylbut-3-yl, 2-cyanoprop-2-yl, 2-methoxycarbonylprop-2-yl or 2-methylaminocarbonylprop-2-yl, 1-alkylthio-2-methylprop-2-yl, 1-alkylsulphinyl-2-methylprop-2-yl, 1-alkylsulphonyl-2-methylprop-2-yl, 2-cyano-1-alkoxyprop-2-yl, 2-methyl-1-[(E and/or Z)-hydroxyimino]-prop-2-yl, 2-methyl-1-[(E and/or Z)-alkoxyimino]-prop-2-yl, 2-methyl-1-[(E and/or Z)-aryloxyimino]-prop-2-yl, 2-methyl-1-[(E and/or Z)-heteroaryloxyimino]-prop-2-yl, 1-alkoxy-prop-2-yl, 1-halo-prop-2-yl, 3-methyl-but-1-yn-3-yl, 1-alkyl-3-methyl-but-1-yn-3-yl, 4-methyl-pent-2-yn-4-yl, 1-hydroxy-4-methyl-pent-2-yn-4-yl, 1-alkoxy-4-methyl-pent-2-yn-4-yl, 1-alkoxyalkoxy-4-methyl-pent-2-yn-4-yl, 1-alkoxyalkoxyalkyl-4-methyl-pent-2-yn-4-yl, 1-cyanoalkyl-3-methylbut-3-yl, 1-haloalkyl-3-methylbut-3-yl, and more preferably, wherein R³ is tert-butyl, 1-halo-2-methylprop-2-yl, 1-fluoro-2-methylprop-2-yl, 1-methoxy-2-methylprop-2-yl, 1-ethoxy-2-methylprop-2-yl, 1-allyloxy-2-methylprop-2-yl, 1-(prop-2-ynyloxy)-2-methylprop-2-yl, 2-cyano-1-ethoxy-prop-2-yl, 2-cyano-1-methoxyprop-2-yl, 1-halo-3-methylbut-3-yl, 1-fluoro-3-methylbut-3-yl, 3-methylbut-1-yn-3-yl, 4-methylpent-2-yn-4-yl, 5-methyl-hex-3-yn-5-yl, 1-methoxy-4-methyl-pent-2-yn-4-yl, 1-allyloxy-4-methyl-pent-2-yn-4-yl, 1-propargyloxy-4-methyl-pent-2-yn-4-yl, 1-ethoxy-4-methyl-pent-2-yn-4-yl.

Another group of preferred compounds of the formula (I) are those, wherein R⁴ is C₁₋₆ alkyl optionally substituted with C₁₋₄alkoxy-(C₁₋₄)alkoxy(C₁₋₄)alkyl, wherein the alkyl group optionally substituted with halo, mono- or di-(C₁₋₆)alkylamino or tri(C₁₋₄)alkylsilyl, or R⁴ is C₁₋₆ alkyl optionally substituted with benzyloxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with C₂₋₆ alkenyloxy or —S(O)ₓ(C₁₋₆)alkyl, wherein x is 0, 1 or 2 and the alkyl group is optionally substituted with halo, mono- or di-(C₁₋₆)alkylamino, —NH(C₁₋₄)alkyl=NOR, wherein R is hydrogen or C₁₋₄alkyl, or wherein the alkyl group is optionally substituted with tri(C₁₋₄)alkylsilyl, or R⁴ is —CR^{uu}═NR^{vv}, wherein R^{uu} is hydrogen or C₁₋₆ alkyl and R^{vv} is hydroxy or optionally substituted C₁₋₆alkoxy.

Another group of preferred compounds of the formula (I) are those, wherein the optionally substituted aryl and optionally substituted heteroaryl rings or moieties of the R₅ values are optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)-alkenyloxy, C₂₋₆ alkynyloxy, halo(C₂₋₆)alkynyloxy, aryl, aryloxy, aryl(C₁₋₆)alkyl, aryl-(C₁₋₆)alkoxy, heteroaryl, heteroaryloxy, heteroaryl(C₁₋₆)alkyl, heteroaryl(C₁₋₆)alkoxy, —SF₅, —S(O)_{g}(C₁₋₄)alkyl wherein g is 0, 1 or 2 and the alkyl is optionally substituted with halo, or R⁵ is optionally substituted with —OSO₂(C₁₋₄)alkyl, wherein the alkyl group is optionally substituted with halo, or R⁵ is optionally substituted with —CONR^{g}R^{h}, —COR^{g}, —CO₂R^{g}, —R^{g}═NR^{h}, —NR^{g}R^{h}, —NR^{g}COR^{h}, —NR^{g}CO₂R^{h}—SO₂NR^{g}R^{h} or —NR^{g}SO₂Rⁱ, wherein R is C₁₋₆ alkyl optionally substituted with halogen and R^{g} and R^{h}, independently of each other, are hydrogen or C₁₋₆ alkyl optionally substituted with halogen, or, in the case of —CONR^{g}R^{h} or —SO₂NR^{g}R^{h}, R^{g}R^{h} may join to form a 5- or 6-membered carbocyclic or heterocyclic ring containing a heteroatom selected from sulphur, oxygen or NR⁰, wherein R⁰ is hydrogen or optionally substituted C₁₋₆alkyl.

Another group of preferred compounds of the formula (I) are those, wherein the optionally substituted aryl, optionally substituted heteroaryl or optionally substituted 5- to 8-membered ring R⁴ is optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)-alkenyloxy, C₂₋₆ alkynyloxy, halo(C₂₋₆)alkynyloxy, —SF₅, —S(O)ₓ(C₁₋₆)alkyl, wherein x is 0, 1 or 2 and the alkyl group is optionally substituted with halo, or R⁴ is optionally substituted with —OSO₂(C₁₋₄)alkyl, wherein the alkyl group is optionally substituted with halogen, —CONR^{x}R^{y}, —CON(OR^{x})R^{y}, —COR^{x}, —CO₂R^{x}, —CR^{x}═NR^{y}, —NR^{x}R^{y}, —NR^{x}COR^{y}, —NR^{x}CO₂R^{y}, —SO₂NR^{x}R^{y} or —NR^{x}SO₂R^{z}, wherein R^{z} is C₁₋₈ alkyl optionally substituted with halogen and R^{x} and R^{y}, independently of each other, are hydrogen or C₁₋₆ alkyl optionally substituted with halogen.

Another group of preferred compounds of the formula (I) are those, wherein R⁵⁰ is C₁₋₄alkyl optionally substituted with halogen, hydroxy, C₁₋₆ alkoxy, C₂₋₆ alkenyl (especially allyl), C₂₋₆ alkynyl (especially propargyl), C₁₋₄ alkoxy(C₁₋₄)alkoxy, cyano, C₁₋₄ alkylcarbonyloxy, aminocarbonyloxy, mono- or di(C₁₋₄)alkylamino-carbonyloxy, S(O)ₚ(C₁₋₆)-alkyl, wherein p is 0, 1 or 2, triazolyl, pyrazolyl, imidazolyl, tri(C₁₋₄)-alkylsilyloxy, optionally substituted phenoxy, optionally substituted thienyloxy, optionally substituted benzyloxy or optionally substituted thienylmethoxy.

Another group of preferred compounds of the formula (I) are those, wherein R⁵⁰ is C₃₋₆cycloalkyl optionally substituted with halogen, hydroxy, C₁₋₆ alkoxy, C₁₋₄ alkoxy(C₁₋₄)alkoxy, cyano, C₁₋₄alkylcarbonyloxy, aminocarbonyloxy, mono- or di(C₁₋₄)alkylamino-carbonyloxy, S(O)ₚ(C₁₋₆)-alkyl, wherein p is 0, 1 or 2, triazolyl, pyrazolyl, imidazolyl, tri(C₁₋₄)-alkylsilyloxy, optionally substituted phenoxy, optionally substituted thienyloxy, optionally substituted benzyloxy or optionally substituted thienylmethoxy.

Another group of preferred compounds of the formula (I) are those, wherein the optionally substituted aryl or the optionally substituted heteroaryl R⁵⁰ is optionally substituted with halogen, hydroxy, mercapto, C₁₋₄alkyl, C₂₋₄alkenyl, C₂₋₄alkynyl, C₁₋₄ alkoxy, C₂₋₄ alkenyloxy, C₂₋₄ alkynyloxy, halo(C₁₋₄)alkyl, halo(C₁₋₄)alkoxy, C₁₋₄ alkylthio, halo(C₁₋₄)-alkylthio, hydroxy(C₁₋₄)alkyl, C₁₋₄alkoxy(C₁₋₄)alkyl, C₃₋₆cycloalkyl, C₃₋₆ cycloalkyl-(C₁₋₄)alkyl, phenoxy, benzyloxy, benzoyloxy, cyano, isocyano, thiocyanato, isothiocyanato, nitro, NR^{p}R^{q}, NHCOR^{p}, —NHCONR^{p}R^{q}, CONR^{p}R^{q}, —SO₂R^{o}, OSO₂R^{o}, COR^{p}, CR^{p}═NR^{q} or N═CR^{p}R^{q}, wherein R^{o} is C₁₋₄alkyl, halo(C₁₋₄)alkyl, C₁₋₄alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄alkyl or C₁₋₄ alkoxy, and R^{p} and R^{q} are independently hydrogen, C₁₋₄alkyl, halo(C₁₋₄)alkyl, C₁₋₄alkoxy, halo(C₁₋₄)alkoxy, C₁₋₄alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄ alkyl or C₁₋₄ alkoxy.

Another group of preferred compounds of the formula (I) are those, wherein L is oxygen.

Another group of preferred compounds of the formula (I) are those, wherein n is 0.

Another group of preferred compounds of the formula (I) are the N-oxides formed by the quinoline moiety in the compounds of the formula I.

Compounds that form part of the invention are illustrated in Tables 1 to 192 below. Melting points (mp) and/or diagnostic molecular ion (e.g. M⁺, [M+1]⁺) values and/or spectroscopic (1H NMR) data are provided in Examples 1-5 while biological activities are provided in Example 6.

TABLE 1
The compounds in Table 1 are of the general formula (I)
where Q1 is chloro, Q2 is hydrogen, Q3 is chloro, n is 0,
L is O, R¹ is methyl, and R² and R³ have the
values given in the table.

| Com- | | |
|---|---|---|
| pound | | |
| No. | R² | R³ |
| 1 | H | CH₃ |
| 2 | CH₃ | CH₃ |
| 3 | H | C₂H₅ |
| 4 | C₂H₅ | C₂H₅ |
| 5 | H | prop-2-yl |
| 6 | CH₃ | prop-2-yl |
| 7 | prop-2-yl | prop-2-yl |
| 8 | CH₃ | n-butyl |
| 9 | H | but-2-yl |
| 10 | H | 2-methyl-prop-1-yl |
| 11 | 2-methyl-prop-1-yl | 2-methyl-prop-1-yl |
| 12 | H | tert-C₄H₉ |
| 13 | CH₃ | tert-C₄H₉ |
| 14 | H | pent-2-yl |
| 15 | H | pent-3-yl |
| 16 | H | 2-methyl-but-2-yl |
| 17 | H | 3-methyl-but-1-yl |
| 18 | H | 3-methyl-pent-2-yl |
| 19 | H | 4-methyl-pent-2-yl |
| 20 | H | 3,3-dimethyl-but-2-yl |
| 21 | H | 2-methyl-hex-2-yl |
| 22 | H | 2,4-dimethyl-pent-2-yl |
| 23 | H | 2,4,4-trimethyl-but-2-yl |
| 24 | H | 2,4,4-trimethyl-pent-2-yl |
| 25 | H | Cl-n-C₃H₆— |
| 26 | H | Cl—CH₂(CH₃)₂C— |
| 27 | H | F₃C(CH₃)₂C— |
| 28 | H | NC—CH₂— |
| 29 | CH₃ | NC—CH₂— |
| 30 | NC—CH₂— | NC—CH₂— |
| 31 | H | (NC)₂CH— |
| 32 | H | NC—C₂H₄— |
| 33 | CH₃ | NC—C₂H₄— |
| 34 | NC—C₂H₄— | NC—C₂H₄— |
| 35 | H | (CH₃)₂C(CN)— |
| 36 | H | C₂H₅(CH₃)C(CN)— |
| 37 | H | (C₂H₅)₂C(CN)— |
| 38 | H | (CH₃)₂CH(CH₃)C(CN)— |
| 39 | H | HO—CH₂(CH₃)₂C— |
| 40 | H | HO—C₂H₄(CH₃)₂C— |
| 41 | H | 1-hydroxy-2-(hydroxymethyl)-prop-2-yl |
| 42 | H | 1-hydroxy-2-(methoxymethyl)prop-2-yl |
| 43 | H | 1-methoxy-2-(methoxymethyl)prop-2-yl |
| 44 | H | 1-hydroxy-2-(hydroxymethyl)-but-2-yl |
| 45 | C₂H₅OC₂H₄— | C₂H₅OC₂H₄— |
| 46 | CH₃ | (CH₃O)₂CHCH₂— |
| 47 | H | CH₃O—CH₂(CH₃)₂C— |
| 48 | H | CH₃O—C₂H₄(CH₃)₂C— |
| 49 | H | C₂H₅O—C₂H₄(CH₃)₂C— |
| 50 | H | CH₃S—CH₂(CH₃)₂C— |
| 51 | H | NC—(CH₃O)CH— |
| 52 | H | CH₃OCH₂(CH₃)C(CN)— |
| 53 | H | CH₃SCH₂(CH₃)C(CN)— |
| 54 | H | CH₃(CO)(CH₃)₂C— |
| 55 | H | CH₃CHBr(CO)(CH₃)₂C— |
| 56 | H | CH₃(CO)(OH)CH(CH₃)₂C— |
| 57 | H | CH₃OC₂H₄(CO)(CH₃)₂C— |
| 58 | H | CH₃(CO)CH₂(CH₃)₂C— |
| 59 | H | CH₃O(CO)(CH₃)CH— |
| 60 | H | CH₃O(CO)(CH₃)₂C— |
| 61 | H | C₂H₅O(CO)C₂H₄— |
| 62 | H | CH₃NH(CO)(CH₃)₂C— |
| 63 | H | (CH₃)₂N(CO)(CH₃)₂C— |
| 64 | H | (CH₃)₃SiCH₂— |
| 65 | H | tert-C₄H₉(CH₃)₂SiO—CH₂(CH₃)₂C— |
| 66 | H | tert-C₄H₉(CH₃)₂SiO—C₂H₄(CH₃)₂C— |
| 67 | H | 4-FPhCH₂OCH₂(CH₃)₂C— |
| 68 | H | C₂H₅OCH₂(CH₃)₂C— |
| 69 | H | CH₃OCH₂CH₂O(CH₃)₂C— |
| 70 | H | CH₂═CHCH₂— |
| 71 | CH₂═CHCH₂— | CH₂═CHCH₂— |
| 72 | H | CH₂═C(CH₃)CH₂— |
| 73 | H | CH₂═CH(CH₃)CH— |
| 74 | H | CH₂═CH(CH₃)₂C— |
| 75 | H | CH₃(CO)CH═CH— |
| 76 | CH₃ | CH₃(CO)CH═CH— |
| 77 | H | pent-3-en-2-yl |
| 78 | H | 2-methyl-hex-3-en-2-yl (E) |
| 79 | H | 2-methyl-hex-3-en-2-yl (Z) |
| 80 | H | 2-methyl-pent-4-en-3-on-2-yl |
| 81 | H | CH₃O(CO)CH═(Cl)C(CH₃)₂C— |
| 82 | H | C₆H₅—C(CH₃)═CH(CH₃)₂C— |
| 83 | CH₂═CHCH₂— | CH₂═CHCH₂OC₂H₄— |
| 84 | H | CH≡CCH₂— |
| 85 | CH₃ | CH≡CCH₂— |
| 86 | H | cycloprop-1-yl |
| 87 | NC—C₂H₄— | cycloprop-1-yl |
| 88 | cycloprop-1-yl | cycloprop-1-yl |
| 89 | H | 1-cyano-cycloprop-1-yl |
| 90 | H | 2-cyano-cycloprop-1-yl |
| 91 | H | 1-methoxycarbonyl-cycloprop-1-yl |
| 92 | H | 1-[N,N-dimethylaminocarbonyl]- |
| | | cycloprop-1-yl |
| 93 | H | 1-[N-methyl-N-methoxy- |
| | | aminocarbonyl]-cycloprop-1-yl |
| 94 | H | 1-cyano-1-cyclopropyl-eth-1-yl |
| 95 | H | cyclopent-1-yl |
| 96 | H | 1-cyano-cyclopent-1-yl |
| 97 | H | cyolohex-1-yl |
| 98 | CH₂═CHCH₂— | cyclohex-1-yl |
| 99 | H | 4-cyano-cyclohex-1-yl |
| 100 | H | 1-cyano-4-methyl-cyolohex-1-yl |
| 101 | H | 4-tert-butyl-1-cyano-cyclohex-1-yl |
| 102 | H | 2-methyl-3-cyanotetrahydro- |
| | | furan-3-yl |
| 103 | H | 5-methyl-1,3-dioxolan-5-yl |
| 104 | H | 5-ethyl-1,3-dioxolan-5-yl |
| 105 | H | 3,5-dimethyl-1,3-dioxolan-5-yl |
| 106 | H | N-ethoxycarbonyl-piperid-4-yl |
| 107 | H | morpholino |
| 108 | H | cyclohex-1-yl-methyl |
| 109 | H | 4-cyano-cyclopenten-3-yl |
| 110 | H | 5-tert-butyl-2H-1,3,4-thiadiazin-2-yl |
| 111 | H | 2-(cyclohexen-1-yl)-eth-1-yl |
| 112 | H | fur-2-yl |
| 113 | H | 5-methoxycarbonyl-fur-2-yl |
| 114 | H | thien-2-yl |
| 115 | H | 2-methoxycarbonyl-thien-3-yl |
| 116 | H | 4-methoxycarbonyl-thien-3-yl |
| 117 | H | oxazol-2-yl |
| 118 | H | 5-methyl-isoxazol-3-yl |
| 119 | H | 4-cyano-3-methyl-isoxazol-5-yl |
| 120 | H | thiazol-2-yl |
| 121 | H | 5-ethylthio-1,3,4-thiadiazol-2-yl |
| 122 | H | fur-2-ylmethyl |
| 123 | H | cyanofur-1-ylmethyl |
| 124 | H | thien-2-ylmethyl |
| 125 | H | C₆H_{5—} |
| 126 | H | 2-Cl—C₆H₄— |
| 127 | H | 2-I—C₆H₄— |
| 128 | H | 2-NC—C₆H₄— |
| 129 | H | 3-CF₃—C₆H₄— |
| 130 | H | 3-CH₃S—C₆H₄— |
| 131 | H | 3-CH₃O(CO)—C₆H₄— |
| 132 | H | 4-Cl—C₆H₄— |
| 133 | H | 4-F—C₆H₄— |
| 134 | H | 4-CF₃O—C₆H₄— |
| 135 | H | 4-(C₂H₅)₂N—C₆H₄— |
| 136 | H | 4-(N-methyl-N-acetyl-amino)-phenyl |
| 137 | H | 2,4-dichlorophenyl |
| 138 | H | 4-methoxy-2-methylphenyl |
| 139 | H | 3,4-dichlorophenyl |
| 140 | H | 3-chloro-4-fluorophenyl |
| 141 | H | 2,5-difluorophenyl |
| 142 | H | 5-fluoro-2-methylphenyl |
| 143 | H | 5,6,7,8-tetrahydronaphth-2-yl |
| 144 | H | 2,3-dihydrobenzofuran-5-yl-methyl |
| 145 | H | 5-cyano-4,6-dimethoxy-pyrid-2-yl |
| 146 | H | 2,6-dimethoxy-pyrid-3-yl |
| 147 | H | 6-chloro-pyridazin-3-yl |
| 148 | H | 4,6-dimethoxy-pyrimid-2-yl |
| 149 | H | 2-chloro-5-fluoro-pyrimid-6-yl |
| 150 | H | C₆H₅CH₂— |
| 151 | CH₃ | C₆H₅CH₂— |
| 152 | H | 2-F—C₆H₄CH₂— |
| 153 | H | 2-Cl—C₆H₄CH₂— |
| 154 | CH₃ | 2-Cl—C₆H₄CH₂— |
| 155 | H | 2-NO₂—C₆H₄CH₂— |
| 156 | H | 2-CH₃—C₆H₄CH₂— |
| 157 | H | 2-CH₃O—C₆H₄CH₂— |
| 158 | H | 2-CHF₂O—C₆H₄CH₂— |
| 159 | H | 2-CH₃S—C₆H₄CH₂— |
| 160 | H | 2-CF₃S—C₆H₄CH₂— |
| 161 | H | 3-Cl—C₆H₄CH₂— |
| 162 | H | 3-I—C₆H₄CH₂— |
| 163 | H | 3-CH₃—C₆H₄CH₂— |
| 164 | H | 3-CH₃O—C₆H₄CH₂— |
| 165 | H | 4-F—C₆H₄CH₂— |
| 166 | H | 4-Cl—C₆H₄CH₂— |
| 167 | H | 4-CH₃—C₆H₄CH₂— |
| 168 | H | 4-CF₃—C₆H₄CH₂— |
| 169 | H | 4-CH₃O—C₆H₄CH₂— |
| 170 | H | 4-CF₃O—C₆H₄CH₂— |
| 171 | H | 2,6-di-F—C₆H₃CH₂— |
| 172 | 3-methyl-but-2-en-1-yl | 2,5-di-F—C₆H₃CH₂— |
| 173 | H | 2-F-4-Cl—C₆H₃CH₂— |
| 174 | H | 2-F-6-Cl—C₆H₃CH₂— |
| 175 | H | 2,6-di-Cl—C₆H₃CH₂— |
| 176 | 4-methyl-pent- | 3,4-di-Cl—C₆H₃CH₂— |
| | 2-en-1-yl | |
| 177 | H | 2-F-6-CH₃O—C₆H₃CH₂— |
| 178 | H | 2,4,5-tri-F—C₆H₂CH₂— |
| 179 | H | 2,4-di-Cl-6-CH₃—C₆H₂CH₂— |
| 180 | H | 3,4,5-tri-CH₃O—C₆H₂CH₂— |
| 181 | H | C₆H₅—CH(CH₃)— |
| 182 | H | 4-F—C₆H₄—CH(CH₃)— |
| 183 | H | 4-NO₂—C₆H₄—CH(CH₃)— |
| 184 | H | 4-n-pentyl-C₆H₄—CH(CH₃)— |
| 185 | H | 4-CH₃SO₂—C₆H₄—CH(CH₃)— |
| 186 | H | C₆H₅(CO)CH₂— |
| 187 | H | C₆H₅—CH(CN)— |
| 188 | H | C₆H₅—(CH₃O)CH— |
| 189 | H | C₆H₅—(CH₃)₂C— |
| 190 | H | m-Cl—C₆H₅—(CH₃)₂C— |
| 191 | H | 3,5-di-Cl—C₆H₃—(CH₃)₂C— |
| 192 | H | C₆H₅—(C₂H₅O(CO))CH— |
| 193 | H | phenethyl |
| 194 | H | 3-methoxy-4-propargyloxy-phenethyl |
| 195 | H | 3-methoxy-4-(pent-2-yn-1-yloxy)- |
| | | phenethyl |
| 196 | H | 2-methyl-3-phenyl-prop-2-yl |
| 197 | H | C₆H₅O—C₂H₄— |
| 198 | H | 4-F—C₆H₄—CH₂OCH₂(CH₃)₂C— |
| 199 | H | C₆H₅—CH₂O(CO)C₂H₄— |
| 200 | H | naphth-2-yl-(CH₃)CH— |
| 201 | NC—C₂H₄— | pyrid-3-ylmethyl |
| 202 | CH₃ | 2-pyrid-2-yleth-1-yl |
| 203 | H | 2-(3-chloro-5-trifluoromethyl- |
| | | pyrid-2-yl)oxyeth-1-yl |
| 204 | H | 2-methyl-4-pyrazin-2-yl- |
| | | but-3-on-2-yl |
| 205 | —(CH₂)₄— | |
| 206 | —(CH₂)₅— | |
| 207 | —(CH₂)₄CH(C₂H₅)— | |
| 208 | —C₃H₆CH[(CO)N(C₂H₅)₂]CH₂— | |
| 209 | —CH(CH₃)CH═CHCH(CH₃)— | |
| 210 | 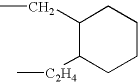 | |
| 211 | —C₂H₄OC₂H₄— | |
| 212 | —CH₂CH(CH₃)OCH(CH₃)CH₂— | |
| 213 | —C₂H₄SCH₂— | |
| 214 | —C₂H₄SC₂H₄— | |
| 215 | —(CH₂)₂NH(CH₂)₂— | |
| 216 | —(CH₂)₂N(p-NO₂—C₆H₄)(CH₂)₂— | |
| 217 | —(CH₂)₂N(m-CF₃—C₆H₄(CH₂)₂— | |
| 218 | —(CH₂)₂N(p-CH₃CO—C₆H₄(CH₂)₂— | |
| 219 | —(CH₂)₂N(pyrid-2-yl)(CH₂)₂— | |
| 220 | H | (H₂C═CHCH₂OCH₂)(CH₃)₂C— |
| 221 | H | (HCCHCH₂OCH₂)(CH₃)₂C— |
| 222 | H | (CH₃CH₂OCH₂)(CH₃)₂C— |
| 223 | H | ((CH₃)₂CHOCH₂)(CH₃)₂C— |
| 224 | H | C₆H₅CH₂OCH₂(CH₃)₂C— |
| 225 | H | (CH₃CH₂OCH₂)(CH₃)₂C— |
| 226 | H | 4-F—C₆H₄—CH₂(CH₃)C(CN)— |
| 227 | H | 4-Cl—C₆H₄—CH₂(CH₃)C(CN)— |
| 228 | H | 4-CH₃O—C₆H₄—CH₂CH₂ |
| | | (CH₃)C(CN)— |
| 229 | H | 2-Cl—C₆H₄—CH₂(CH₃)C(CN)— |
| 230 | H | (CH₃)₂CH—CH₂(CH₃)C(CN)— |
| 231 | H | 1-methoxymethyl-cycloprop-1-yl |
| 232 | H | 1-benzyloxymethyl-cycloprop-1-yl |
| 233 | H | 1-methoxymethoxy-2-methyl-prop-2-yl |
| 235 | H | 1-cyclopropyl-eth-1-yl |
| 236 | H | 2-fluoro-eth-1-yl |
| 237 | H | 2,2,2-trifluoro-1-methyl-eth-1-yl |
| 238 | H | HC≡CC(CH₃)₂— |
| 239 | CH₃ | HC≡CC(CH₃)₂— |
| 240 | H | HC≡CC(CH₂CH₃)(CH₃)— |
| 241 | CH₃ | HC≡CC(CH₂CH₃)(CH₃)— |
| 242 | H | HC≡CC(CH₂CH₂)— |
| 243 | CH₃ | HC≡CC(CH₂CH₂)— |
| 244 | H | (H₃C)C≡CC(CH₃)₂— |
| 245 | CH₃ | (H₃C)C≡CC(CH₃)₂— |
| 246 | H | (H₃C)C≡CC(CH₂CH₃)(CH₃)— |
| 247 | CH₃ | (H₃C)C≡CC(CH₂CH₃)(CH₃)— |
| 248 | H | (H₃C)C≡CC(CH₂CH₂)— |
| 249 | CH₃ | (H₃C)C≡CC(CH₂CH₂)— |
| 250 | H | (HOCH₂)C≡CC(CH₃)₂— |
| 251 | H | (CH₃OCH₂)C≡CC(CH₃)₂— |
| 252 | H | (HOCH₂)C≡CC(CH₃)(CH₂CH₃)₂— |
| 253 | H | (CH₃CH₂OCH₂)C≡CC(CH₃)₂— |
| 254 | H | (CH₃OCH₂)C≡CC(CH₃)(CH₂CH₃)₂— |
| 255 | H | (CH₃OC₂H₄OC₂H₄)C≡CC(CH₃)₂— |
| 256 | H | (Cl-n-C₃H₆)C≡CC(CH₃)₂— |
| 257 | H | (NC-n-C₃H₆)C≡CC(CH₃)₂— |
| 258 | H | (CH₃SCH₂)C≡CC(CH₃)₂— |
| 259 | H | (C₆H₅)C≡CC(CH₃)₂— |
| 260 | H | (CH₃)₂(CH₃O)CC≡CC(CH₃)₂— |
| 261 | H | (H₂C═CHCH₂OCH₂)(CH₃)CH— |
| 262 | H | (HC≡CHCH₂OCH₂)(CH₃)CH— |
| 263 | H | (CH₃CH₂OCH₂)(CH₃)CH— |
| 264 | H | (CH₃OCH₂)(CH₃)CH— |
| 265 | H | ((CH₃)₂CHOCH₂)(CH₃)CH— |
| 266 | H | C₆H₅CH₂OCH₂(CH₃)CH— |
| 267 | H | (CH₃CH₂OCH₂)(CH₃)CH— |
| 268 | H | (cC₄H₇)CH— |
| 269 | H | (cC₄H₇)CH₃C— |
| 270 | H | FCH₂(CH₃)CH— |
| 271 | H | ClCH₂(CH₃)CH— |
| 272 | H | FCH₂CH₂(CH₃)CH— |
| 273 | H | ClCH₂CH₂(CH₃)CH— |
| 274 | H | FCH₂(CH₃)₂C— |
| 275 | H | FCH₂CH₂(CH₃)₂C— |
| 276 | H | ClCH₂CH₂(CH₃)₂C— |
| 277 | H | CH₃O(C₂H₄C≡CC(CH₃)₂— |
| 278 | H | tetrahydro-furan-2-ylmethyl |
| 279 | H | 1-(tetrahydro-furan-2-yl)ethyl |
| 280 | H | 1-methyl-1-(tetrahydro-furan-2-yl)ethyl |
| 281 | H | 2-[1,3]dioxolan-2-yl-ethyl |
| 282 | H | 2-[1,3]dioxolan-2-yl-1-methyl-ethyl |
| 283 | H | 2-[1,3]dioxolan-2-yl-1,1-dimethyl-ethyl |
| 284 | H | prop-1-yl |
| 285 | CH₃ | prop-1-yl |
| 286 | H | thiophen-3-ylmethyl |
| 287 | H | 1-(thiophen-3-yl)-eth-1-yl |
| 289 | H | 1-methyl-1-(thiophen-3-yl)-eth-1-yl |
| 290 | H | cyclopent-1-yl |
| 291 | H | 3-F—C₆H₄—CH₂— |
| 292 | H | 3-F—C₆H₄—CH(CH₃)— |
| 293 | H | 3-F—C₆H₄—C(CH₃)₂— |
| 294 | H | C₂H₅C≡CC(CH₃)₂— |
| 294 | H | nC₃H₇C≡CC(CH₂CH₃)(CH₃)— |
| 296 | H | i-C₃H₇C≡CC(CH₂CH₂)— |
| 297 | H | n-C₄H₉C≡CC(CH₃)₂— |
| 298 | H | sec-C₄H₉C≡CC(CH₃)₂— |
| 299 | H | iso-C₄H₉C≡CC(CH₃)₂— |
| 300 | H | tert-C₄H₉C≡CC(CH₃)₂— |
| 301 | H | HOC₂H₄C≡CC(CH₃)₂— |
| 302 | H | CH₃(CH₃O)(CH)C≡CC(CH₃)₂— |
| 303 | H | (nC₃H₇OCH₂)C≡CC(CH₃)₂— |
| 304 | H | (nC₃H₇OCH₂CH₂)C≡CC(CH₃)₂— |
| 305 | H | (tert-C₄H₉OCH₂)C≡CC(CH₃)₂— |
| 306 | H | (tert-C₄H₉OCH₂CH₂)C≡CC(CH₃)₂— |
| 307 | H | (NCCH₂)C≡CC(CH₃)₂— |
| 308 | H | (NCCH₂CH₂)C≡CC(CH₃)₂— |
| 309 | H | (C₆H₅OCH₂)C≡CC(CH₃)₂— |
| 310 | H | (C₆H₅OCH₂CH₂)C≡CC(CH₃)₂— |
| 311 | H | (4-FC₆H₅)C≡CC(CH₃)₂— |
| 312 | H | (4-ClC₆H₅)C≡CC(CH₃)₂— |
| 313 | H | (4-BrC₆H₅)C≡CC(CH₃)₂— |
| 314 | H | (4-CH₃—C₆H₅)C≡CC(CH₃)₂— |
| 315 | H | (3-FC₆H₅)C≡CC(CH₃)₂— |
| 316 | H | (3-ClC₆H₅)C≡CC(CH₃)₂— |
| 317 | H | (3-CH₃—C₆H₅)C≡CC(CH₃)₂— |
| 318 | H | (2-FC₆H₅)C≡CC(CH₃)₂— |
| 319 | H | (2-ClC₆H₅)C≡CC(CH₃)₂— |
| 320 | H | (2-BrC₆H₅)C≡CC(CH₃)₂— |
| 321 | H | (2-CH₃—C₆H₅)C≡CC(CH₃)₂— |
| 322 | H | (thien-2-yl)C≡CC(CH₃)₂— |
| 323 | H | (thien-3-yl)C≡CC(CH₃)₂— |

### Table 2

The compounds in Table 2 are of the general formula (I) where Q1 is bromo, Q2 is hydrogen, Q3 is chloro, n is 0, L is O, R¹ is methyl, R² and R³ have the values listed in Table 1. Thus, compound 1 of Table 2 is the same as compound 1 of Table 1 except that in compound 1 of Table 2 Q1 is bromo, Q2 is hydrogen, Q3 is chloro. Similarly, compounds 2 to 323 of Table 2 are the same as compounds 2 to 323 of Table 1, respectively, except that in the compounds of Table 2 Q1 is bromo, Q2 is hydrogen, Q3 is chloro.

### Table 3

The compounds in Table 3 are of the general formula (I) where Q1 is iodo, Q2 is hydrogen, Q3 is chloro, n is 0, L is O, R¹ is methyl, R² and R³ have the values listed in Table 1. Thus, compound 1 of Table 3 is the same as compound 1 of Table 1 except that in compound 1 of Table 3 Q1 is iodo, Q2 is hydrogen, Q3 is chloro. Similarly, compounds 2 to 323 of Table 3 are the same as compounds 2 to 323 of Table 1, respectively, except that in the compounds of Table 3 Q1 is iodo, Q2 is hydrogen, Q3 is chloro.

### Table 4

The compounds in Table 4 are of the general formula (I) where Q1 is fluoro, Q2 is hydrogen, Q3 is chloro, n is 0, L is O, R¹ is methyl, R² and R³ have the values listed in Table 1. Thus, compound 1 of Table 4 is the same as compound 1 of Table 1 except that in compound 1 of Table 4 Q1 is fluoro, Q2 is hydrogen, Q3 is chloro. Similarly, compounds 2 to 323 of Table 4 are the same as compounds 2 to 323 of Table 1, respectively, except that in the compounds of Table 3 Q1 is fluoro, Q2 is hydrogen, Q3 is chloro.

### Table 5

The compounds in Table 5 are of the general formula (I) where Q1 is chloro, Q2 is hydrogen, Q3 is fluoro, n is 0, L is O, R¹ is methyl, R² and R³ have the values listed in Table 1. Thus, compound 1 of Table 5 is the same as compound 1 of Table 1 except that in compound 1 of Table 5 Q1 is chloro, Q2 is hydrogen, Q3 is fluoro. Similarly, compounds 2 to 323 of Table 5 are the same as compounds 2 to 323 of Table 1, respectively, except that in the compounds of Table 5 Q1 is chloro, Q2 is hydrogen, Q3 is fluoro.

### Table 6

The compounds in Table 6 are of the general formula (I) where Q1 is bromo, Q2 is hydrogen, Q3 is fluoro, n is 0, L is O, R¹ is methyl, R² and R³ have the values listed in Table 1. Thus, compound 1 of Table 6 is the same as compound 1 of Table 1 except that in compound 1 of Table 5 Q1 is bromo, Q2 is hydrogen, Q3 is fluoro. Similarly, compounds 2 to 323 of Table 6 are the same as compounds 2 to 323 of Table 1, respectively, except that in the compounds of Table 6 Q1 is bromo, Q2 is hydrogen, Q3 is fluoro.

### Table 7

The compounds in Table 7 are of the general formula (I) where Q1 is iodo, Q2 is hydrogen, Q3 is fluoro, n is 0, L is O, R¹ is methyl, R² and R³ have the values listed in Table 1. Thus, compound 1 of Table 7 is the same as compound 1 of Table 1 except that in compound 1 of Table 7 Q1 is iodo, Q2 is hydrogen, 03 is fluoro. Similarly, compounds 2 to 323 of Table 7 are the same as compounds 2 to 323 of Table 1, respectively, except that in the compounds of Table 7 Q1 is iodo, Q2 is hydrogen, Q3 is fluoro.

### Table 8

The compounds in Table 8 are of the general formula (I) where Q1 is fluoro, Q2 is hydrogen, Q3 is fluoro, n is 0, L is O, R¹ is methyl, R² and R³ have the values listed in Table 1. Thus, compound 1 of Table 8 is the same as compound 1 of Table 1 except that in compound 1 of Table 8 Q1 is fluoro, Q2 is hydrogen, Q3 is fluoro. Similarly, compounds 2 to 323 of Table 8 are the same as compounds 2 to 323 of Table 1, respectively, except that in the compounds of Table 8 Q1 is fluoro, Q2 is hydrogen, Q3 is fluoro.

### Table 9

The compounds in Table 9 are of the general formula (I) where Q1 is hydrogen, Q2 is hydrogen, Q3 is chloro, n is 0, L is O, R¹ is methyl, R² and R³ have the values listed in Table 1. Thus, compound 1 of Table 9 is the same as compound 1 of Table 1 except that in compound 1 of Table 9 Q1 is hydrogen, Q2 is hydrogen, Q3 is chloro. Similarly, compounds 2 to 323 of Table 9 are the same as compounds 2 to 323 of Table 1, respectively, except that in the compounds of Table 9 Q1 is hydrogen, Q2 is hydrogen, Q3 is chloro.

### Table 10

The compounds in Table 10 are of the general formula (I) where Q1 is hydrogen, Q2 is hydrogen, Q3 is fluoro, n is 0, L is O, R¹ is methyl, R² and R³ have the values listed in Table 1. Thus, compound 1 of Table 10 is the same as compound 1 of Table 1 except that in compound 1 of Table 10 Q1 is hydrogen, Q2 is hydrogen, Q3 is fluoro. Similarly, compounds 2 to 323 of Table 10 are the same as compounds 2 to 323 of Table 1, respectively, except that in the compounds of Table 10 Q1 is hydrogen, Q2 is hydrogen, Q3 is fluoro.

### Table 11

The compounds in Table 11 are of the general formula (I) where Q1 is hydrogen, Q2 is hydrogen, Q3 is bromo, n is 0, L is O, R¹ is methyl, R² and R³ have the values listed in Table 1. Thus, compound 1 of Table 11 is the same as compound 1 of Table 1 except that in compound 1 of Table 11 Q1 is hydrogen, Q2 is hydrogen, Q3 is bromo. Similarly, compounds 2 to 323 of Table 11 are the same as compounds 2 to 323 of Table 1, respectively, except that in the compounds of Table 11 Q1 is hydrogen, Q2 is hydrogen, Q3 is bromo.

### Table 12

The compounds in Table 12 are of the general formula (I) where Q1 is chloro, Q2 is hydrogen, Q3 is methyl, n is 0, L is O, R¹ is methyl, R² and R³ have the values listed in Table 1. Thus, compound 1 of Table 12 is the same as compound 1 of Table 1 except that in compound 1 of Table 12 Q1 is chloro, Q2 is hydrogen, Q3 is methyl. Similarly, to compounds 2 to 323 of Table 12 are the same as compounds 2 to 323 of Table 1, respectively, except that in the compounds of Table 12 Q1 is chloro, Q2 is hydrogen, Q3 is methyl.

### Table 13

The compounds in Table 13 are of the general formula (I) where Q1 is bromo, Q2 is hydrogen, Q3 is methyl, n is 0, L is O, R¹ is methyl, R² and R³ have the values listed in Table 1. Thus, compound 1 of Table 13 is the same as compound 1 of Table 1 except that in compound 1 of Table 13 Q1 is bromo, Q2 is hydrogen, Q3 is methyl. Similarly, compounds 2 to 323 of Table 13 are the same as compounds 2 to 323 of Table 1, respectively, except that in the compounds of Table 13 Q1 is bromo, Q2 is hydrogen, Q3 is methyl.

### Table 14

The compounds in Table 14 are of the general formula (I) where Q1 is iodo, Q2 is hydrogen, Q3 is methyl, n is 0, L is O, R¹ is methyl, R² and R³ have the values listed in Table 1. Thus, compound 1 of Table 14 is the same as compound 1 of Table 1 except that in compound 1 of Table 14 Q1 is iodo, Q2 is hydrogen, Q3 is methyl. Similarly, compounds 2 to 323 of Table 14 are the same as compounds 2 to 323 of Table 1, respectively, except that in the compounds of Table 14 Q1 is iodo, Q2 is hydrogen, Q3 is methyl.

### Table 15

The compounds in Table 15 are of the general formula (I) where Q1 is fluoro, Q2 is hydrogen, Q3 is methyl, n is 0, L is O, R¹ is methyl, R² and R³ have the values listed in Table 1. Thus, compound 1 of Table 15 is the same as compound 1 of Table 1 except that in compound 1 of Table 15 Q1 is fluoro, Q2 is hydrogen, Q3 is methyl. Similarly, compounds 2 to 323 of Table 15 are the same as compounds 2 to 323 of Table 1, respectively, except that in the compounds of Table 15 Q1 is fluoro, Q2 is hydrogen, Q3 is methyl.

### Table 16

The compounds in Table 16 are of the general formula (I) where Q1 is hydrogen, Q2 is hydrogen, Q3 is methyl, n is 0, L is O, R¹ is methyl, R² and R³ have the values listed in Table 1. Thus, compound 1 of Table 16 is the same as compound 1 of Table 1 except that in compound 1 of Table 16 Q1 is hydrogen, Q2 is hydrogen, Q3 is methyl. Similarly, compounds 2 to 323 of Table 16 are the same as compounds 2 to 323 of Table 1, respectively, except that in the compounds of Table 16 Q1 is hydrogen, Q2 is hydrogen, Q3 is methyl.

### Table 17

The compounds in Table 17 are of the general formula (I) where Q1 is chloro, Q2 is hydrogen, Q3 is chloro, n is 0, L is O, R¹ is ethyl, R² and R³ have the values listed in Table 1. Thus, compound 1 of Table 17 is the same as compound 1 of Table 1 except that in compound 1 of Table 17 R¹ is ethyl, Q1 is chloro, Q2 is hydrogen, Q3 is chloro. Similarly, compounds 2 to 323 of Table 17 are the same as compounds 2 to 323 of Table 1, respectively, except that in the compounds of Table 17 R¹ is ethyl, Q1 is chloro, Q2 is hydrogen, Q3 is chloro.

### Table 18

The compounds in Table 18 are of the general formula (I) where Q1 is bromo, Q2 is hydrogen, Q3 is chloro, n is 0, L is O, R¹ is ethyl, R² and R³ have the values listed in Table 1. Thus, compound 1 of Table 18 is the same as compound 1 of Table 1 except that in compound 1 of Table 18 R¹ is ethyl, Q1 is bromo, Q2 is hydrogen, Q3 is chloro. Similarly, compounds 2 to 323 of Table 18 are the same as compounds 2 to 323 of Table 1, respectively, except that in the compounds of Table 18 R¹ is ethyl, Q1 is bromo, Q2 is hydrogen, Q3 is chloro.

### Table 19

The compounds in Table 19 are of the general formula (I) where Q1 is iodo, Q2 is hydrogen, Q3 is chloro, n is 0, L is O, R¹ is ethyl, R² and R³ have the values listed in Table 1. Thus, compound 1 of Table 19 is the same as compound 1 of Table 1 except that in compound 1 of Table 19 R¹ is ethyl, Q1 is iodo, Q2 is hydrogen, Q3 is chloro. Similarly, compounds 2 to 323 of Table 19 are the same as compounds 2 to 323 of Table 1, respectively, except that in the compounds of Table 19 R¹ is ethyl, Q1 is iodo, Q2 is hydrogen, Q3 is chloro.

### Table 20

The compounds in Table 20 are of the general formula (I) where Q1 is chloro, Q2 is hydrogen, Q3 is fluoro, n is 0, L is O, R¹ is ethyl, R² and R³ have the values listed in Table 1. Thus, compound 1 of Table 20 is the same as compound 1 of Table 1 except that in compound 1 of Table 20 R¹ is ethyl, Q1 is chloro, Q2 is hydrogen, Q3 is fluoro. Similarly, compounds 2 to 323 of Table 20 are the same as compounds 2 to 323 of Table 1, respectively, except that in the compounds of Table 20 R¹ is ethyl, Q1 is chloro, Q2 is hydrogen, Q3 is fluoro.

### Table 21

The compounds in Table 21 are of the general formula (I) where Q1 is bromo, Q2 is hydrogen, Q3 is fluoro, n is 0, L is O, R¹ is ethyl, R² and R³ have the values listed in Table 1. Thus, compound 1 of Table 21 is the same as compound 1 of Table 1 except that in compound 1 of Table 21 R¹ is ethyl, Q1 is bromo, Q2 is hydrogen, Q3 is fluoro. Similarly, compounds 2 to 323 of Table 21 are the same as compounds 2 to 323 of Table 1, respectively, except that in the compounds of Table 21 R¹ is ethyl, Q1 is bromo, Q2 is hydrogen, Q3 is fluoro.

### Table 22

The compounds in Table 22 are of the general formula (I) where Q1 is iodo, Q2 is hydrogen, Q3 is fluoro, n is 0, L is O, R¹ is ethyl, R² and R³ have the values listed in Table 1. Thus, compound 1 of Table 22 is the same as compound 1 of Table 1 except that in compound 1 of Table 22 R¹ is ethyl, Q1 is iodo, Q2 is hydrogen, Q3 is fluoro. Similarly, compounds 2 to 323 of Table 22 are the same as compounds 2 to 323 of Table 1, respectively, except that in the compounds of Table 22 R¹ is ethyl, Q1 is iodo, Q2 is hydrogen, Q3 is fluoro.

### Table 23

The compounds in Table 23 are of the general formula (I) where Q1 is hydrogen, Q2 is hydrogen, Q3 is chloro, n is 0, L is O, R¹ is ethyl, R² and R³ have the values listed in Table 1. Thus, compound 1 of Table 23 is the same as compound 1 of Table 1 except that in compound 1 of Table 23 R¹ is ethyl, Q1 is hydrogen, Q2 is hydrogen, Q3 is chloro. Similarly, compounds 2 to 323 of Table 23 are the same as compounds 2 to 323 of Table 1, respectively, except that in the compounds of Table 23 R¹ is ethyl, Q1 is hydrogen, Q2 is hydrogen, Q3 is chloro.

### Table 24

The compounds in Table 24 are of the general formula (I) where Q1 is hydrogen, Q2 is hydrogen, Q3 is fluoro, n is 0, L is O, R¹ is ethyl, R² and R³ have the values listed in Table 1. Thus, compound 1 of Table 24 is the same as compound 1 of Table 1 except that in compound 1 of Table 24 R¹ is ethyl, Q1 is hydrogen, Q2 is hydrogen, Q3 is fluoro. Similarly, compounds 2 to 323 of Table 24 are the same as compounds 2 to 323 of Table 1, respectively, except that in the compounds of Table 24 R¹ is ethyl, Q1 is hydrogen, Q2 is hydrogen, Q3 is fluoro.

### Table 25

The compounds in Table 25 are of the general formula (I) where Q1 is chloro, Q2 is hydrogen, Q3 is methyl, n is 0, L is O, R¹ is ethyl, R² and R³ have the values listed in Table 1. Thus, compound 1 of Table 25 is the same as compound 1 of Table 1 except that in compound 1 of Table 25 R¹ is ethyl, Q1 is chloro, Q2 is hydrogen, Q3 is methyl. Similarly, compounds 2 to 323 of Table 25 are the same as compounds 2 to 323 of Table 1, respectively, except that in the compounds of Table 25 R¹ is ethyl, Q1 is chloro, Q2 is hydrogen, Q3 is methyl.

### Table 26

The compounds in Table 26 are of the general formula (I) where Q1 is bromo, Q2 is hydrogen, Q3 is methyl, n is 0, L is O, R¹ is ethyl, R² and R³ have the values listed in Table 1. Thus, compound 1 of Table 26 is the same as compound 1 of Table 1 except that in compound 1 of Table 26 R¹ is ethyl, Q1 is bromo, Q2 is hydrogen, Q3 is methyl. Similarly, compounds 2 to 323 of Table 26 are the same as compounds 2 to 323 of Table 1, respectively, except that in the compounds of Table 26 R¹ is ethyl, Q1 is bromo, Q2 is hydrogen, Q3 is methyl.

### Table 27

The compounds in Table 27 are of the general formula (I) where Q1 is iodo, Q2 is hydrogen, Q3 is methyl, n is 0, L is O, R¹ is ethyl, R² and R³ have the values listed in Table 1. Thus, compound 1 of Table 27 is the same as compound 1 of Table 1 except that in compound 1 of Table 27 R¹ is ethyl, Q1 is iodo, Q2 is hydrogen, Q3 is methyl. Similarly, compounds 2 to 323 of Table 27 are the same as compounds 2 to 323 of Table 1, respectively, except that in the compounds of Table 27 R¹ is ethyl, Q1 is iodo, Q2 is hydrogen, Q3 is methyl.

### Table 28

The compounds in Table 28 are of the general formula (I) where Q1 is hydrogen, Q2 is hydrogen, Q3 is methyl, n is 0, L is O, R¹ is ethyl, R² and R³ have the values listed in Table 1. Thus, compound 1 of Table 28 is the same as compound 1 of Table 1 except that in compound 1 of Table 28 R¹ is ethyl, Q1 is hydrogen, Q2 is hydrogen, Q3 is methyl. Similarly, compounds 2 to 323 of Table 28 are the same as compounds 2 to 323 of Table 1, respectively, except that in the compounds of Table 28 R¹ is ethyl, Q1 is hydrogen, Q2 is hydrogen, Q3 is methyl.

### Table 29

The compounds in Table 29 are of the general formula (I) where Q1 is thiophen-3-yl, Q2 is hydrogen, Q3 is chloro, n is 0, L is O, R¹ is methyl, R² and R³ have the values listed in Table 1. Thus, compound 1 of Table 29 is the same as compound 1 of Table 1 except that in compound 1 of Table 29 Q1 is thiophen-3-yl, Q2 is hydrogen, Q3 is chloro. Similarly, compounds 2 to 323 of Table 29 are the same as compounds 2 to 323 of Table 1, respectively, except that in the compounds of Table 29 Q1 is thiophen-3-yl, Q2 is hydrogen, Q3 is chloro.

### Table 30

The compounds in Table 30 are of the general formula (I) where Q1 is thiophen-3-yl, Q2 is hydrogen, Q3 is fluoro, n is 0, L is O, R¹ is methyl, R² and R³ have the values listed in Table 1. Thus, compound 1 of Table 30 is the same as compound 1 of Table 1 except that in compound 1 of Table 30 Q1 is thiophen-3-yl, Q2 is hydrogen, Q3 is fluoro. Similarly, compounds 2 to 323 of Table 30 are the same as compounds 2 to 323 of Table 1, respectively, except that in the compounds of Table 30 Q¹ is thiophen-3-yl, Q2 is hydrogen, Q3 is fluoro.

### Table 31

The compounds in Table 31 are of the general formula (I) where Q1 is thiophen-2-yl, Q2 is hydrogen, Q3 is chloro, n is 0, L is O, R¹ is methyl, R² and R³ have the values listed in Table 1. Thus, compound 1 of Table 31 is the same as compound 1 of Table 1 except that in compound 1 of Table 31 Q1 is thiophen-2-yl, Q2 is hydrogen, Q3 is chloro. Similarly, compounds 2 to 323 of Table 31 are the same as compounds 2 to 323 of Table 1, respectively, except that in the compounds of Table 31 Q1 is thiophen-2-yl, Q2 is hydrogen, Q3 is chloro.

### Table 32

The compounds in Table 32 are of the general formula (I) where Q1 is thiophen-2-yl, Q2 is hydrogen, Q3 is fluoro, n is 0, L is O, R¹ is methyl, R² and R³ have the values listed in Table 1. Thus, compound 1 of Table 32 is the same as compound 1 of Table 1 except that in compound 1 of Table 32 Q1 is thiophen-2-yl, Q2 is hydrogen, Q3 is fluoro. Similarly, compounds 2 to 323 of Table 32 are the same as compounds 2 to 323 of Table 1, respectively, except that in the compounds of Table 32 Q1 is thiophen-2-yl, Q2 is hydrogen, Q3 is fluoro.

### Table 33

The compounds in Table 33 are of the general formula (I) where Q1 is thiophen-3-yl, Q2 is hydrogen, Q3 is chloro, n is 0, L is O, R¹ is ethyl, R² and R³ have the values listed in Table 1. Thus, compound 1 of Table 33 is the same as compound 1 of Table 1 except that in compound 1 of Table 33 Q1 is thiophen-3-yl, Q2 is hydrogen, Q3 is chloro. Similarly, compounds 2 to 323 of Table 33 are the same as compounds 2 to 323 of Table 1, respectively, except that in the compounds of Table 33 Q1 is thiophen-3-yl, Q2 is hydrogen, Q3 is chloro.

### Table 34

The compounds in Table 34 are of the general formula (I) where Q1 is thiophen-3-yl, Q2 is hydrogen, Q3 is fluoro, n is 0, L is O, R¹ is ethyl, R² and R³ have the values listed in Table 1. Thus, compound 1 of Table 34 is the same as compound 1 of Table 1 except that in compound 1 of Table 34 Q1 is thiophen-3-yl, Q2 is hydrogen, Q3 is fluoro. Similarly, compounds 2 to 323 of Table 34 are the same as compounds 2 to 323 of Table 1, respectively, except that in the compounds of Table 34 Q1 is thiophen-3-yl, Q2 is hydrogen, Q3 is fluoro.

### Table 35

The compounds in Table 35 are of the general formula (I) where Q1 is thiophen-2-yl, Q2 is hydrogen, Q3 is chloro, n is 0, L is O, R¹ is ethyl, R² and R³ have the values listed in Table 1. Thus, compound 1 of Table 35 is the same as compound 1 of Table 1 except that in compound 1 of Table 35 Q1 is thiophen-2-yl, Q2 is hydrogen, Q3 is chloro. Similarly, compounds 2 to 323 of Table 35 are the same as compounds 2 to 323 of Table 1, respectively, except that in the compounds of Table 35 Q1 is thiophen-2-yl, Q2 is hydrogen, Q3 is chloro.

### Table 36

The compounds in Table 36 are of the general formula (I) where Q1 is thiophen-2-yl, Q2 is hydrogen, Q3 is fluoro, n is 0, L is O, R¹ is ethyl, R² and R³ have the values listed in Table 1. Thus, compound 1 of Table 36 is the same as compound 1 of Table 1 except that in compound 1 of Table 36 Q1 is thiophen-2-yl, Q2 is hydrogen, Q3 is fluoro. Similarly, compounds 2 to 323 of Table 36 are the same as compounds 2 to 323 of Table 1, respectively, except that in the compounds of Table 36 Q1 is thiophen-2-yl, Q2 is hydrogen, Q3 is fluoro.

### Table 37

The compounds in Table 37 are of the general formula (I) where Q1 is chloro, Q2 is methyl, Q3 is chloro, n is 0, L is O, R¹ is methyl, R² and R³ have the values listed in Table 1. Thus, compound 1 of Table 37 is the same as compound 1 of Table 1 except that in compound 1 of Table 37 Q1 is chloro, Q2 is methyl, Q3 is chloro. Similarly, compounds 2 to 323 of Table 37 are the same as compounds 2 to 323 of Table 1, respectively, except that in the compounds of Table 37 Q1 is chloro, Q2 is methyl, Q3 is chloro,

### Table 38

The compounds in Table 38 are of the general formula (I) where Q1 is bromo, Q2 is methyl, Q3 is chloro, n is 0, L is O, R¹ is methyl, R² and R³ have the values listed in Table 1. Thus, compound 1 of Table 38 is the same as compound 1 of Table 1 except that in compound 1 of Table 38 Q1 is bromo, Q2 is methyl, Q3 is chloro. Similarly, compounds 2 to 323 of Table 38 are the same as compounds 2 to 323 of Table 1, respectively, except that in the compounds of Table 38 Q1 is bromo, Q2 is methyl, Q3 is chloro.

### Table 39

The compounds in Table 39 are of the general formula (I) where Q1 is iodo, Q2 is methyl, Q3 is chloro, n is 0, L is O, R¹ is methyl, R² and R³ have the values listed in Table 1. Thus, compound 1 of Table 39 is the same as compound 1 of Table 1 except that in compound 1 of Table 39 Q1 is iodo, Q2 is methyl, Q3 is chloro. Similarly, compounds 2 to 323 of Table 39 are the same as compounds 2 to 323 of Table 1, respectively, except that in the compounds of Table 39 Q1 is iodo, Q2 is methyl, Q3 is chloro.

### Table 40

The compounds in Table 40 are of the general formula (I) where Q1 is fluoro, Q2 is methyl, Q3 is chloro, n is 0, L is O, R¹ is methyl, R² and R³ have the values listed in Table 1. Thus, compound 1 of Table 40 is the same as compound 1 of Table 1 except that in compound 1 of Table 40 Q1 is fluoro, Q2 is methyl, Q3 is chloro. Similarly, compounds 2 to 323 of Table 40 are the same as compounds 2 to 323 of Table 1, respectively, except that in the compounds of Table 40 Q1 is fluoro, Q2 is methyl, Q3 is chloro.

### Table 41

The compounds in Table 41 are of the general formula (I) where Q1 is chloro, Q2 is methyl, Q3 is fluoro, n is 0, L is O, R¹ is methyl, R² and R³ have the values listed in Table 1. Thus, compound 1 of Table 41 is the same as compound 1 of Table 1 except that in compound 1 of Table 41 Q1 is chloro, Q2 is methyl, Q3 is fluoro. Similarly, compounds 2 to 323 of Table 41 are the same as compounds 2 to 323 of Table 1, respectively, except that in the compounds of Table 41 Q1 is chloro, Q2 is methyl, Q3 is fluoro.

### Table 42

The compounds in Table 42 are of the general formula (I) where Q1 is bromo, Q2 is methyl, Q3 is fluoro, n is 0, L is O, R¹ is methyl, R² and R³ have the values listed in Table 1. Thus, compound 1 of Table 42 is the same as compound 1 of Table 1 except that in compound 1 of Table 42 Q1 is bromo, Q2 is methyl, Q3 is fluoro. Similarly, compounds 2 to 323 of Table 42 are the same as compounds 2 to 323 of Table 1, respectively, except that in the compounds of Table 42 Q1 is bromo, Q2 is methyl, Q3 is fluoro.

### Table 43

The compounds in Table 43 are of the general formula (I) where Q1 is iodo, Q2 is methyl, Q3 is fluoro, n is 0, L is O, R¹ is methyl, R² and R³ have the values listed in Table 1. Thus, compound 1 of Table 43 is the same as compound 1 of Table 1 except that in compound 1 of Table 43 Q1 is iodo, Q2 is methyl, Q3 is fluoro. Similarly, compounds 2 to 323 of Table 43 are the same as compounds 2 to 323 of Table 1, respectively, except that in the compounds of Table 43 Q1 is iodo, Q2 is methyl, Q3 is fluoro.

### Table 44

The compounds in Table 44 are of the general formula (I) where Q1 is fluoro, Q2 is methyl, 03 is fluoro, n is 0, L is O, R¹ is methyl, R² and R³ have the values listed in Table 1. Thus, compound 1 of Table 44 is the same as compound 1 of Table 1 except that in compound 1 of Table 44 Q1 is fluoro, Q2 is methyl, Q3 is fluoro. Similarly, compounds 2 to 323 of Table 44 are the same as compounds 2 to 323 of Table 1, respectively, except that in the compounds of Table 44 Q1 is fluoro, Q2 is methyl, Q3 is fluoro.

### Table 45

The compounds in Table 45 are of the general formula (I) where Q1 is chloro, Q2 is methyl, Q3 is bromo, n is 0, L is O, R¹ is methyl, R² and R³ have the values listed in Table 1. Thus, compound 1 of Table 45 is the same as compound 1 of Table 1 except that in compound 1 of Table 45 Q1 is chloro, Q2 is methyl, Q3 is bromo. Similarly, compounds 2 to 323 of Table 45 are the same as compounds 2 to 323 of Table 1, respectively, except that in the compounds of Table 45 Q1 is chloro, Q2 is methyl, Q3 is bromo.

### Table 46

The compounds in Table 46 are of the general formula (I) where Q1 is bromo, Q2 is methyl, Q3 is bromo, n is 0, L is O, R¹ is methyl, R² and R³ have the values listed in Table 1. Thus, compound 1 of Table 46 is the same as compound 1 of Table 1 except that in compound 1 of Table 46 Q1 is bromo, Q2 is methyl, Q3 is bromo. Similarly, compounds 2 to 323 of Table 46 are the same as compounds 2 to 323 of Table 1, respectively, except that in the compounds of Table 46 Q1 is bromo, Q2 is methyl, Q3 is bromo.

### Table 47

The compounds in Table 47 are of the general formula (I) where Q1 is iodo, Q2 is methyl, Q3 is bromo, n is 0, L is O, R¹ is methyl, R² and R³ have the values listed in Table 1. Thus, compound 1 of Table 47 is the same as compound 1 of Table 1 except that in compound 1 of Table 47 Q1 is iodo, Q2 is methyl, Q3 is bromo. Similarly, compounds 2 to 323 of Table 47 are the same as compounds 2 to 323 of Table 1, respectively, except that in the compounds of Table 47 Q1 is iodo, Q2 is methyl, Q3 is bromo.

### Table 48

The compounds in Table 48 are of the general formula (I) where Q1 is fluoro, Q2 is methyl, Q3 is bromo, n is 0, L is O, R¹ is methyl, R² and R³ have the values listed in Table 1. Thus, compound 1 of Table 48 is the same as compound 1 of Table 1 except that in compound 1 of Table 48 Q1 is fluoro, Q2 is methyl, Q3 is bromo. Similarly, compounds 2 to 323 of Table 48 are the same as compounds 2 to 323 of Table 1, respectively, except that in the compounds of Table 48 Q1 is fluoro, Q2 is methyl, Q3 is bromo.

### Table 49

The compounds in Table 49 are of the general formula (I) where Q1 is chloro, Q2 is chloro, Q3 is fluoro, n is 0, L is O, R¹ is methyl, R² and R³ have the values listed in Table 1. Thus, compound 1 of Table 49 is the same as compound 1 of Table 1 except that in compound 1 of Table 49 Q1 is chloro, Q2 is chloro, Q3 is fluoro. Similarly, compounds 2 to 323 of Table 49 are the same as compounds 2 to 323 of Table 1, respectively, except that in the compounds of Table 49 Q1 is Q1 is chloro, Q2 is chloro, Q3 is fluoro.

### Table 50

The compounds in Table 50 are of the general formula (I) where Q1 is bromo, Q2 is chloro, Q3 is fluoro, n is 0, L is O, R¹ is methyl, R² and R³ have the values listed in Table 1. Thus, compound 1 of Table 50 is the same as compound 1 of Table 1 except that in compound 1 of Table 50 Q1 is bromo, Q2 is chloro, Q3 is fluoro. Similarly, compounds 2 to 323 of Table 50 are the same as compounds 2 to 323 of Table 1, respectively, except that in the compounds of Table 50 Q1 is Q1 is bromo, Q2 is chloro, Q3 is fluoro.

### Table 51

The compounds in Table 51 are of the general formula (I) where Q1 is fluoro, Q2 is chloro, Q3 is fluoro, n is 0, L is O, R¹ is methyl, R² and R³ have the values listed in Table 1. Thus, compound 1 of Table 51 is the same as compound 1 of Table 1 except that in compound 1 of Table 51 Q1 is fluoro, Q2 is chloro, Q3 is fluoro. Similarly, compounds 2 to 323 of Table 51 are the same as compounds 2 to 323 of Table 1, respectively, except that in the compounds of Table 51 Q1 is Q1 is fluoro, Q2 is chloro, Q3 is fluoro.

### Table 52

The compounds in Table 52 are of the general formula (I) where Q1 is iodo, Q2 is chloro, Q3 is fluoro, n is 0, L is O, R¹ is methyl, R² and R³ have the values listed in Table 1. Thus, compound 1 of Table 52 is the same as compound 1 of Table 1 except that in compound 1 of Table 52 Q1 is iodo, Q2 is chloro, Q3 is fluoro. Similarly, compounds 2 to 323 of Table 52 are the same as compounds 2 to 323 of Table 1, respectively, except that in the compounds of Table 52 Q1 is Q1 is iodo, Q2 is chloro, Q3 is fluoro.

### Table 53

The compounds in Table 53 are of the general formula (I) where Q1 is chloro, Q2 is bromo, Q3 is fluoro, n is 0, L is O, R¹ is methyl, R² and R³ have the values listed in Table 1. Thus, compound 1 of Table 53 is the same as compound 1 of Table 1 except that in compound 1 of Table 5301 is chloro, Q2 is bromo, Q3 is fluoro. Similarly, compounds 2 to 323 of Table 53 are the same as compounds 2 to 323 of Table 1, respectively, except that in the compounds of Table 53 Q1 is Q1 is chloro, Q2 is bromo, Q3 is fluoro.

### Table 54

The compounds in Table 54 are of the general formula (I) where Q1 is bromo, Q2 is bromo, Q3 is fluoro, n is 0, L is O, R¹ is methyl, R² and R³ have the values listed in Table 1. Thus, compound 1 of Table 54 is the same as compound 1 of Table 1 except that in compound 1 of Table 54 Q1 is bromo, Q2 is bromo, Q3 is fluoro. Similarly, compounds 2 to 323 of Table 54 are the same as compounds 2 to 323 of Table 1, respectively, except that in the compounds of Table 54 Q1 is Q1 is bromo, Q2 is bromo, Q3 is fluoro.

### Table 55

The compounds in Table 55 are of the general formula (I) where Q1 is fluoro, Q2 is bromo, Q3 is fluoro, n is 0, L is O, R¹ is methyl, R² and R³ have the values listed in Table 1. Thus, compound 1 of Table 55 is the same as compound 1 of Table 1 except that in compound 1 of Table 55 Q1 is fluoro, Q2 is bromo, Q3 is fluoro. Similarly, compounds 2 to 323 of Table 55 are the same as compounds 2 to 323 of Table 1, respectively, except that in the compounds of Table 55 Q1 is Q1 is fluoro, Q2 is bromo, Q3 is fluoro.

### Table 56

The compounds in Table 56 are of the general formula (I) where Q1 is iodo, Q2 is bromo, Q3 is fluoro, n is 0, L is O, R¹ is methyl, R² and R³ have the values listed in Table 1. Thus, compound 1 of Table 56 is the same as compound 1 of Table 1 except that in compound 1 of Table 56 Q1 is iodo, Q2 is bromo, Q3 is fluoro. Similarly, compounds 2 to 323 of Table 56 are the same as compounds 2 to 323 of Table 1, respectively, except that in the compounds of Table 56 Q1 is Q1 is iodo, Q2 is bromo, Q3 is fluoro.

### Table 57

The compounds in Table 57 are of the general formula (I) where Q1 is chloro, Q2 is fluoro, Q3 is fluoro, n is 0, L is O, R¹ is methyl, R² and R³ have the values listed in Table 1. Thus, compound 1 of Table 57 is the same as compound 1 of Table 1 except that in compound 1 of Table 57 Q1 is chloro, Q2 is fluoro, Q3 is fluoro. Similarly, compounds 2 to 323 of Table 57 are the same as compounds 2 to 323 of Table 1, respectively, except that in the compounds of Table 57 Q1 is Q1 is chloro, Q2 is fluoro, Q3 is fluoro.

### Table 58

The compounds in Table 58 are of the general formula (I) where Q1 is bromo, Q2 is fluoro, Q3 is fluoro, n is 0, L is O, R¹ is methyl, R² and R³ have the values listed in Table 1. Thus, compound 1 of Table 58 is the same as compound 1 of Table 1 except that in compound 1 of Table 58 Q1 is bromo, Q2 is fluoro, Q3 is fluoro. Similarly, compounds 2 to 323 of Table 58 are the same as compounds 2 to 323 of Table 1, respectively, except that in the compounds of Table 58 Q1 is Q1 is bromo, Q2 is fluoro, Q3 is fluoro.

### Table 59

The compounds in Table 59 are of the general formula (I) where Q1 is fluoro, Q2 is fluoro, Q3 is fluoro, n is 0, L is O, R¹ is methyl, R² and R³ have the values listed in Table 1. Thus, compound 1 of Table 59 is the same as compound 1 of Table 1 except that in compound 1 of Table 59 Q1 is fluoro, Q2 is fluoro, Q3 is fluoro. Similarly, compounds 2 to 323 of Table 59 are the same as compounds 2 to 323 of Table 1, respectively, except that in the compounds of Table 59 Q1 is Q1 is fluoro, Q2 is fluoro, Q3 is fluoro.

### Table 60

The compounds in Table 60 are of the general formula (I) where Q1 is iodo, Q2 is fluoro, Q3 is fluoro, n is 0, L is O, R¹ is methyl, R² and R³ have the values listed in Table 1. Thus, compound 1 of Table 60 is the same as compound 1 of Table 1 except that in compound 1 of Table 60 Q1 is iodo, Q2 is fluoro, Q3 is fluoro. Similarly, compounds 2 to 323 of Table 60 are the same as compounds 2 to 323 of Table 1, respectively, except that in the compounds of Table 60 Q1 is Q1 is iodo, Q2 is fluoro, Q3 is fluoro.

### Table 61

The compounds in Table 61 are of the general formula (I) where Q1 is thiophen-3-yl, Q2 is fluoro, Q3 is fluoro, n is 0, L is O, R¹ is methyl, R² and R³ have the values listed in Table 1. Thus, compound 1 of Table 61 is the same as compound 1 of Table 1 except that in compound 1 of Table 61 Q1 is thiophen-3-yl, Q2 is fluoro, Q3 is fluoro. Similarly, compounds 2 to 323 of Table 61 are the same as compounds 2 to 323 of Table 1, respectively, except that in the compounds of Table 61 Q1 is Q1 is thiophen-3-yl, Q2 is fluoro, Q3 is fluoro.

### Tables 62 to 121

Tables 62 to 121 correspond exactly to Tables 1 to 61 (i.e. Table 62 corresponds exactly to Table 1, Table 63 corresponds exactly to Table 2, and so on) the only difference being that in each of Tables 62 to 121, L is S instead of O.

### Tables 122 to 181

Tables 122 to 181 correspond exactly to Tables 1 to 61 (i.e. Table 122 corresponds exactly to Table 1, Table 123 corresponds exactly to Table 2, and so on) the only difference being that in each of Tables 122 to 181, n is 1 instead of 0.

### Tables 182 to 241

Tables 182 to 241 correspond exactly to Tables 1 to 61 (i.e. Table 182 corresponds exactly to Table 1, Table 183 corresponds exactly to Table 2, and so on) the only difference being that in each of Tables 182 to 241, n is 2 instead of 0.

The compounds of general formula (I) may be prepared as outlined in Schemes 1 to 8 below, in which Q1, Q2, Q3, R¹, R² and R³ have the meanings given above, R¹⁴ is H or C₁₋₄ alkyl, as indicated, R¹⁰ is C₁₋₆ alkyl, optionally substituted benzyl, optionally substituted C₂₋₆ alkenyl, optionally substituted C₂₋₄ alkynyl, R⁶, R⁷, R⁸, R⁹, R¹² and R¹³ are independently H or C₁₋₄ alkyl, R^{g} is H or C₁₋₃ alkyl, R^{h} is H or C₁₋₃ alkyl, Rⁱ is C₁₋₆ alkyl, optionally substituted benzyl, optionally substituted C₂₋₆ alkenyl, optionally substituted C₂₋₄ alkynyl, m is 0, 1 or 2, DMF is N,N-dimethylformamide, NBS is N-bromosuccinimide, NCS is N-chlorosuccinimide and MCPBA is m-chloroperbenzoic acid. Other abbreviations are defined in the text.

Where typical or preferred process conditions (reaction temperature, time, solvent, mole ratios of reactants) are given, unless otherwise stated other process conditions can also be used. While optimum reaction conditions may vary with the particular reactants or solvents used, such conditions can be determined by routine optimisation procedures by one skilled in the art.

Compounds of formula (1), where n is 0 and L is O, may be prepared as shown in Scheme 1. Esters of formula (2), where R⁵ is C₁₋₄ alkyl, may be halogenated to give haloesters of formula (3), where Hal is a halogen atom such as bromine, chlorine or iodine, by reaction with a halogenating agent such as N-bromosuccinimide, in a suitable solvent such as carbon tetrachloride or acetonitrile, in the presence of a radical initiator such as AIBN (azo-isobutyronitrile), and a light source, at between ambient temperature and the reflux temperature of the solvent. Compounds of general formula (3) are then reacted with alkanethiols of general formula R¹SH, in the presence of a base such as sodium hydride, in a suitable solvent such as DMF, to give compounds of general formula (6), or are reacted with alkanethiol salts R¹S⁻M⁺, where M is a metal such as sodium or lithium, in a suitable solvent such as DMF, to give compounds of general formula (6).

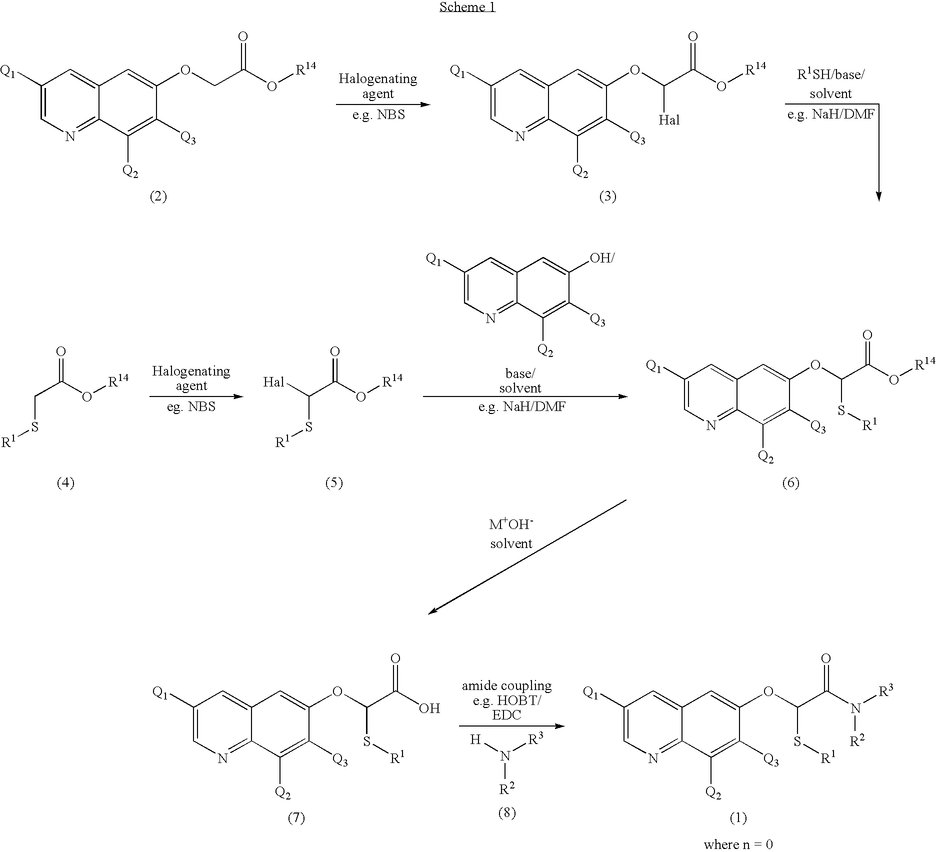

Alternatively esters of general formula (4) are halogenated to give haloesters of formula (5), where Hal is a halogen atom such as bromine, chlorine or iodine, by reaction with a halogenating agent such as N-chlorosuccinimide or N-bromosuccinimide, in a suitable solvent such as carbon tetrachloride or acetonitrile, at between 0° C. and the reflux temperature of the solvent. Haloesters of formula (5) are reacted with 6-hydroxy quinolines, where Q1, Q2 and Q3 are as defined above, in the presence of a base such as potassium t-butoxide, potassium carbonate, or sodium hydride in a suitable solvent such as t-butanol, 1,4-dioxane or DMF, at between ambient temperature and the reflux temperature of the solvent, to give compounds of formula (6). Compounds of formula (6) are hydrolysed to acids of formula (7) by reaction with an alkali metal hydroxide M⁺OH⁻, in a suitable solvent such as aqueous methanol, ethanol, or THF (tetrahydrofuran) at between ambient temperature and the reflux temperature of the solvent followed by acidification. Acids of formula (7) can be condensed with amines of formula (8), using suitable activating agents such as HOBT (1-hydroxybenzotriazole) and EDC (1-ethyl-3-N,N-dimethylaminopropylcarbodiimide hydrochloride), at between 0° C. and ambient temperature in a suitable solvent such as DMF, to give compounds of general formula (1) where n is 0 and L is O.

Compounds of general formula (1), where n is 1 or 2, are prepared by oxidation of compounds (1) where n=0 to the sulphoxide (n is 1) or sulphone (n is 2) oxidation state, as shown in Scheme 2. For example, esters of the general formula (6) where R⁵ is C₁₋₄ alkyl can be oxidised to sulphoxides of formula (9) with an oxidising agent such as sodium periodate in a suitable solvent such ethanol, between 0° C. and ambient temperature. Sulphones of formula (10) can be made either directly from compounds of formula (6) with two or more equivalents of an oxidising agent such as m-chloro-perbenzoic acid (MCPBA), in a suitable solvent such as dichloromethane between 0° C. and the reflux temperature of the solvent, or from sulphoxides of formula (9) with one or more equivalents of m-chloroperbenzoic acid. Sulfides of formula (6), sulphoxides of formula (9) or sulphones of formula (10) can be hydrolysed to the corresponding acids (7), (11) or (12) by reaction with an alkali metal hydroxide in a suitable solvent such as ethanol at between 0° C. and the reflux temperature of the solvent followed by acidification. The acids of formula (7), (11) or (12) can be condensed with amines of formula (8), using suitable activating agents such as HOBT and EDC, at between 0° C. and ambient temperature, to give compounds of general formula (1) where n is 0, 1 or 2.

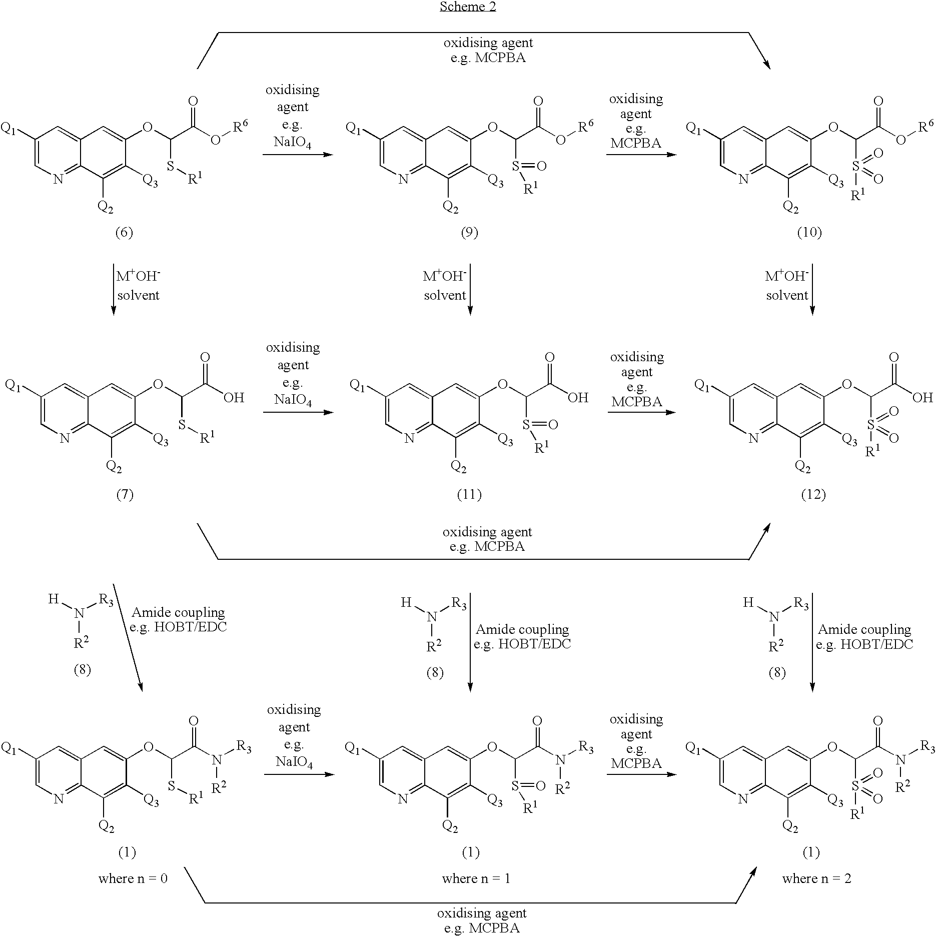

Similarly, sulphoxides of formula (11) and of formula (1) where n is 1 can be prepared from sulfides of formula (7) and of formula (1) where n is 0 respectively, using oxidising agents such as sodium metaperiodate or m-chloroperbenzoic acid as described above. Sulphones of formula (12) and of formula (1) where n is 2, can be prepared either from sulfides of formula (7) and of formula (1) where n is 0, by using at least two equivalents of oxidising agents such as m-chloroperbenzoic acid, or from sulphoxides of formula (11) and of formula (1) where n is 1, using one or more equivalents of oxidising agents such as m-chloroperbenzoic acid, as described above.

Compounds of formula (1) can also be prepared as shown in Scheme 3. Acids of formula (13) can be condensed with amines of formula (8), using suitable activating agents such as HOBT and EDC, at between 0° C. and ambient temperature, to give compounds of formula (14). Compounds of formula (14) can be halogenated to compounds of formula (16) using a halogenating agent such as N-chlorosuccinimide, in to a suitable solvent such as carbon tetrachloride or acetonitrile, at between 0° C. and ambient temperature. Amides of formula (16) can also be prepared from acid halides of formula (15) by reaction with amines of formula (8) in the presence of a base such as triethylamine in a suitable solvent such as dichloromethane, at between 0° C. and ambient temperature.

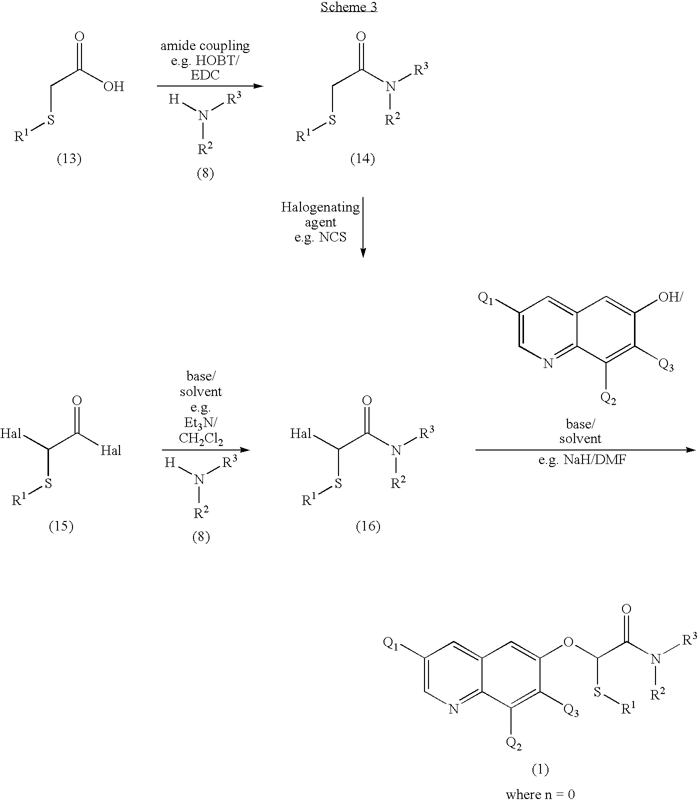

Halosulphides of formula (16) can be reacted with substituted 6-hydroxy quinolines, in the presence of a base such as potassium carbonate or sodium hydride, in a suitable solvent such as DMF, at between 0° C. and 80° C., to give compounds of formula (1) where n is 0.

As shown in Scheme 4, amines of the general formula (18) or (20), which are examples of amines of the general formula (8) wherein R² is H, may be prepared by alkylation of an aminoalcohol of the general formula (17) or (19) using a suitable base, such as n-butyl lithium or sodium hydride, followed by reaction with a suitable alkylating reagent R¹⁰LG, such as an alkyl iodide, for example, methyl iodide, to form an alkylated compound of the general formula (18) or (20), respectively. A carbonyl derivative R¹²COR¹³ (21), for example formaldehyde, can be reacted with ammonia, usually in form of ammonium chloride, and cyanide, conveniently in form of an aqueous solution sodium cyanide, to provide an α-aminoalkyne (22) (Strecker synthesis).

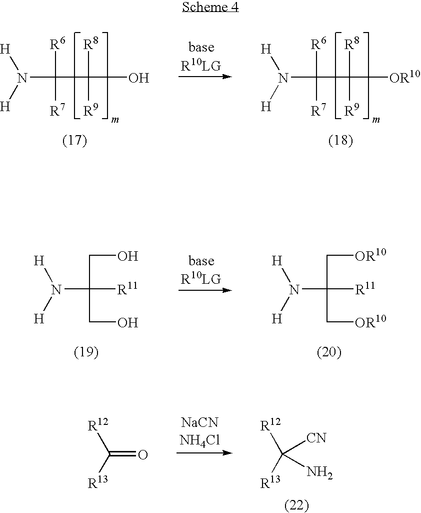

As shown in Scheme 5, silyl-protected aminoalkynes of the general formula (24) may be obtained by reacting amines of general formula (23) with 1,2-bis-(chlorodimethylsilyl)-ethane in the presence of a suitable base, such as a tertiary organic amine base, for example, triethylamine. Amines of the general formula (26), which are examples of amines of the general formula (8) wherein R² is H and R³ is —(CR³⁰R⁴⁰)C≡CR⁵⁰, may be prepared by alkylation of a silyl-protected aminoalkyne of the general formula (24) using a suitable base, such as n-butyl lithium, followed by reaction with a suitable alkylating reagent R⁵⁰LG, such as an alkyl iodide, for example, methyl iodide, to form an alkylated compound of the general formula (25). The silyl protecting group may then be removed from a compound of the general formula (25) with, for example, an aqueous acid to form an aminoalkyne of the general formula (26).

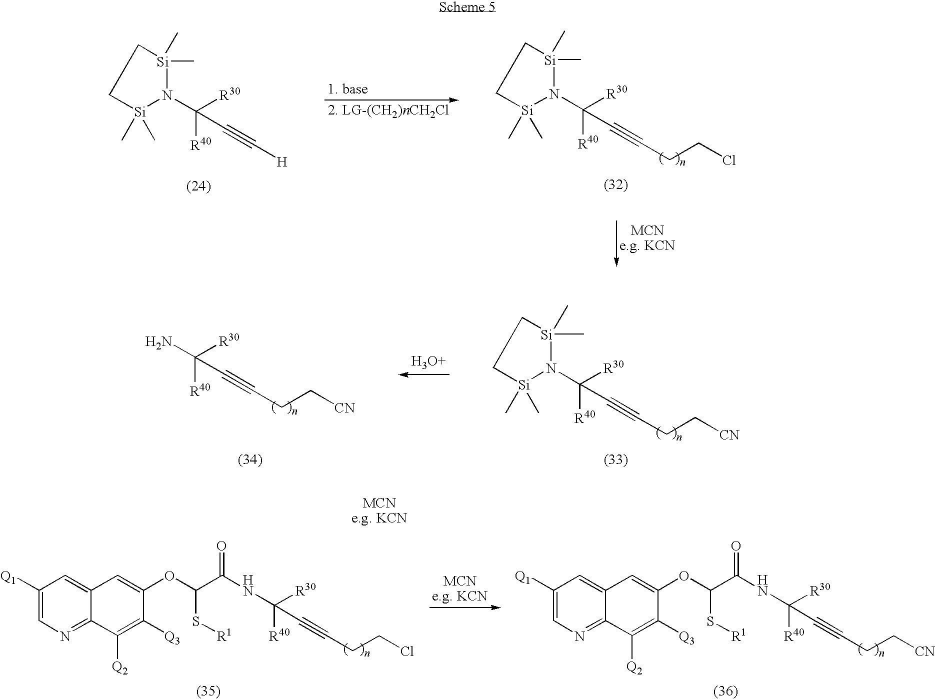

In a similar procedure, a silyl-protected aminoalkyne of the general formula (24) may be reacted with a carbonyl derivative R^{a}COR^{b}, for example formaldehyde, using a suitable base, such as n-butyl lithium, to provide an aminoalkyne (27) containing a hydroxyalkyl moiety. A compound of the general formula (27) may either first be treated with a base, such as sodium hydride or potassium bis(trimethylsilyl)amide followed by a compound R^{c}LG, where LG represents a leaving group such as a halogen, or sulphonate ester such as OSO₂Me, or OSO₂-4-tolyl, for example ethyl iodide, to give a compound of the general formula (29). After removal of the silyl protecting group, compounds of general formula (30) are obtained. Alternatively, the silyl protecting group can first be removed to yield compounds of the general formula (28). Aminoalkynes of the general formula (28) may be further derivatised by reacting with a silylating agent, for example t-butyl-dimethylsilyl chloride, to give a derivative silylated on oxygen of the general formula (31).

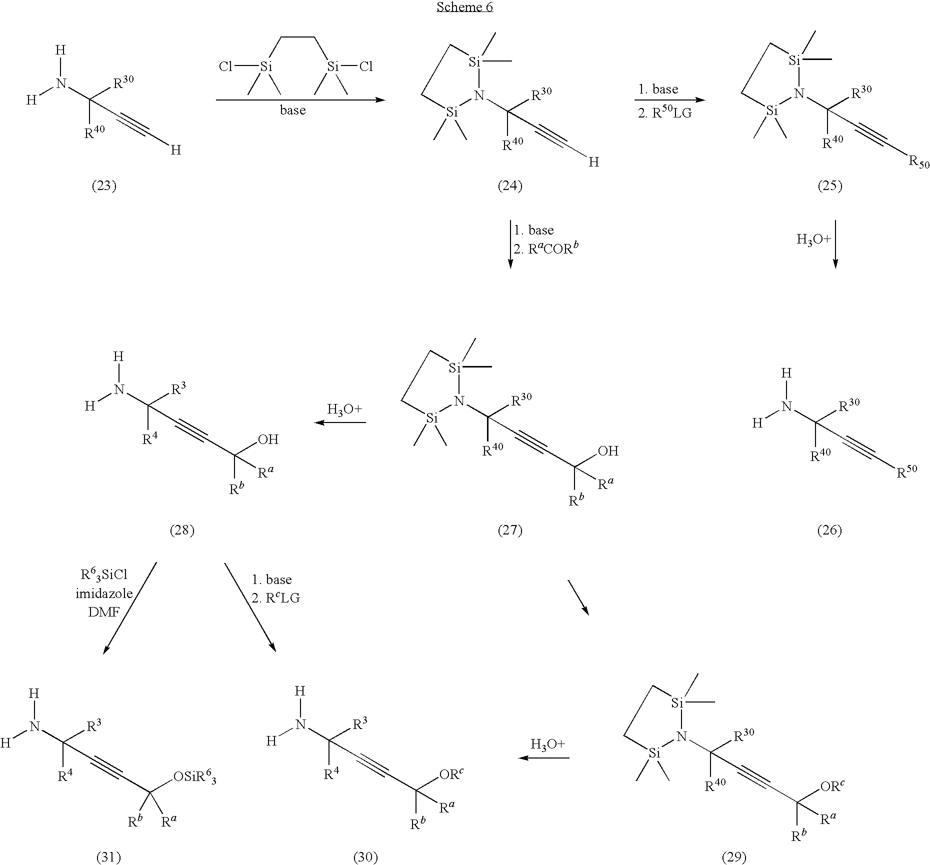

As shown in Scheme 6, silyl-protected aminoalkynes of the general formula (32) may be obtained by reacting silyl-protected amines of general formula (24) with chloroalkanes bearing a suitable leaving group, for example bromide or iodide, in the presence of a suitable base, such as sodium or lithium amide base, for example, sodium bis(trimethylsilyl)amide or sodium amide. Amines of the general formula (34), which are examples of amines of the general formula (8) wherein R² is H and R³ is —(CR³⁰R⁴⁰)C≡CR⁵⁰ may be prepared by displacement of chloride anion by cyanide, followed by removal of the silyl protecting group with, for example, an aqueous acid, to form a cyano compound of the general formula (34).

In a similar procedure, an amide of the general formula (35) can be reacted with, for example, potassium cyanide yielding a cyano amidoalkyne of the general formula (36).

As shown in Scheme 7, compounds of the general formula (1), wherein R⁵⁰ is H, may be reacted under Sonogashira conditions with, for example, optionally substituted aryl or heteroaryl chlorides, bromides, iodides or triflates to form substituted aryl or heteroaryl compounds of general formula (1), wherein R⁵⁰ is an optionally substituted aryl or heteroaryl group. A suitable palladium catalyst is bis(triphenylphosphine)palladium (11) chloride.

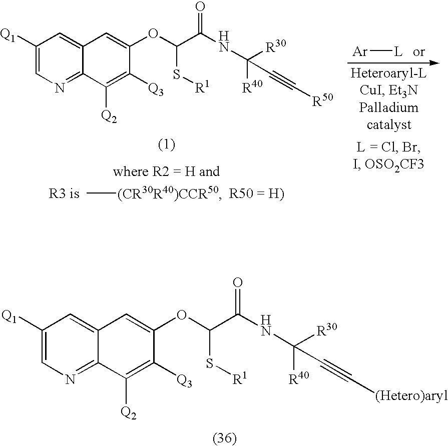

Other amines of the general formula (8) are either commercially available or may be prepared by standard literature methods or standard modifications.

As shown in Scheme 8, compounds of the general formula (1), wherein Q1 is bromine or iodine, may be reacted under Suzuki conditions with, for example, optionally substituted aryl or heteroaryl boronic acids to form substituted aryl or heteroaryl compounds of general formula (1), wherein Q1 is an optionally substituted aryl or heteroaryl group. A suitable palladium catalyst is tetrakis(triphenylphosphine)palladium(0).

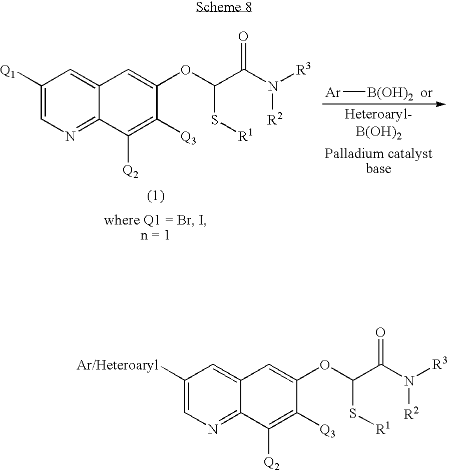

Thioamides (Compounds of the general formula (1) where L=S) may be prepared from the corresponding amides using thionating agents such as phosphous pentasulphide, Lawesson's or Davy's reagents or prepared from the corresponding thionoacids or thionoesters using standard literature methods or standard modifications.

The substituted 6-hydroxy quinolines are available, or may be prepared using straightforward techniques of organic chemistry. When the compounds are not commercially available, they may be prepared from available precursors using straightforward transformations that are well known in the art that are well described in standard textbooks of heterocyclic chemistry. For example, substituted aromatic amines may be readily converted into substituted quinolin-6-ols with appropriate electrophiles, such as 2,2,3 tribromopropanal. Examples of such reactions are provided in Examples 1-3.

The compounds of formula (I) are active fungicides and may be used to control one or more of the following pathogens: Pyricularia oryzae (Magnaporthe grisea) on rice and wheat and other Pyricularia spp. on other hosts; Puccinia triticina (or recondita), Puccinia striiformis and other rusts on wheat, Puccinia hordei, Puccinia striiformis and other rusts on barley, and rusts on other hosts (for example turf, rye, coffee, pears, apples, peanuts, sugar beet, vegetables and ornamental plants); Erysiphe cichoracearum on cucurbits (for example melon); Blumeria (or Erysiphe) graminis (powdery mildew) on barley, wheat, rye and turf and other powdery mildews on various hosts, such as Sphaerotheca macularis on hops, Sphaerotheca fusca (Sphaerotheca fuliginea) on cucurbits (for example cucumber), Leveillula taurica on tomatoes, aubergine and green pepper, Podosphaera leucotricha on apples and Uncinula necator on vines; Cochliobolus spp., Helminthosporium spp., Drechslera spp. (Pyrenophora spp.), Rhynchosporium spp., Mycosphaerella graminicola (Septoria tritici) and Phaeosphaeria nodorum (Stagonospora nodorum or Septoria nodorum), Pseudocercosporella herpotrichoides and Gaeumannomyces graminis on cereals (for example wheat, barley, rye), turf and other hosts; Cercospora arachidicola and Cercosporidium personatum on peanuts and other Cercospora spp. on other hosts, for example sugar beet, bananas, soya beans and rice; Botrytis cinerea (grey mould) on tomatoes, strawberries, vegetables, vines and other hosts and other Botrytis spp. on other hosts; Alternaria spp. on vegetables (for example carrots), oil-seed rape, apples, tomatoes, potatoes, cereals (for example wheat) and other hosts; Venturia spp. (including Venturia inaequalis (scab)) on apples, pears, stone fruit, tree nuts and other hosts; Cladosporium spp. on a range of hosts including cereals (for example wheat) and tomatoes; Monilinia spp. on stone fruit, tree nuts and other hosts; Didymella spp. on tomatoes, turf, wheat, cucurbits and other hosts; Phoma spp. on oil-seed rape, turf, rice, potatoes, wheat and other hosts; Aspergillus spp. and Aureobasidium spp. on wheat, lumber and other hosts; Ascochyta spp. on peas, wheat, barley and other hosts; Stemphylium spp. (Pleospora spp.) on apples, pears, onions and other hosts; summer diseases (for example bitter rot (Glomerella cingulata), black rot or frogeye leaf spot (Botryosphaeria obtusa), Brooks fruit spot (Mycosphaerella pomi), Cedar apple rust (Gymnosporangium juniperi-virginianae), sooty blotch (Gloeodes pomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. on soybeans, tobacco, onions and other hosts, Pseudoperonospora humuli on hops and Pseudoperonospora cubensis on cucurbits; Pythium spp. (including Pythium ultimum) on turf and other hosts; Phytophthora infestans on potatoes and tomatoes and other Phytophthora spp. on vegetables, strawberries, avocado, pepper, ornamentals, tobacco, cocoa and other hosts; Thanatephorus cucumeris on rice and turf and other Rhizoctonia spp. on various hosts such as wheat and barley, peanuts, vegetables, cotton and turf; Sclerotinia spp. on turf, peanuts, potatoes, oil-seed rape and other hosts; Sclerotium spp. on turf, peanuts and other hosts; Gibberella fujikuroi on rice; Colletotrichum spp. on a range of hosts including turf, coffee and vegetables; 5 Laetisaria fuciformis on turf; Mycosphaerella spp. on bananas, peanuts, citrus, pecans, papaya and other hosts; Diaporthe spp. on citrus, soybean, melon, pears, lupin and other hosts; Elsinoe spp. on citrus, vines, olives, pecans, roses and other hosts; Verticillium spp. on a range of hosts including hops, potatoes and tomatoes; Pyrenopeziza spp. on oil-seed rape and other hosts; Oncobasidium theobromae on cocoa causing vascular streak dieback; Fusarium spp., Typhula spp., Microdochium nivale, Ustilago spp., Urocystis spp., Tilletia spp. and Claviceps purpurea on a variety of hosts but particularly wheat, barley, turf and maize; Ramularia spp. on sugar beet, barley and other hosts; post-harvest diseases particularly of fruit (for example Penicillium digitatum, Penicillium italicum and Trichoderma viride on oranges, Colletotrichum musae and Gloeosporium musarum on bananas and Botrytis cinerea on grapes); other pathogens on vines, notably Eutypa lata, Guignardia bidwellii, Phellinus igniarus, Phomopsis viticola, Pseudopeziza tracheiphila and Stereum hirsutum; other pathogens on trees (for example Lophodermium seditiosum) or lumber, notably Cephaloascus fragrans, Ceratocystis spp., Ophiostoma piceae, Penicillium spp., Trichoderma pseudokoningii, Trichoderma viride, Trichoderma harzianum, Aspergillus niger, Leptographium lindbergi and Aureobasidium pullulans; and fungal vectors of viral diseases (for example Polymyxa graminis on cereals as the vector of barley yellow mosaic virus (BYMV) and Polymyxa betae on sugar beet as the vector of rhizomania).

A compound of formula (1) may move acropetally, basipetally or locally in plant tissue to be active against one or more fungi. Moreover, a compound of formula (1) may be volatile enough to be active in the vapour phase against one or more fungi on the plant.

The invention therefore provides a method of combating or controlling phytopathogenic fungi which comprises applying a fungicidally effective amount of a compound of formula (I), or a composition containing a compound of formula (I), to a plant, to a seed of a plant, to the locus of the plant or seed or to soil or any other plant growth medium, e.g. nutrient solution.

The term “plant” as used herein includes seedlings, bushes and trees. Furthermore, the fungicidal method of the invention includes protectant, curative, systemic, eradicant and antisporulant treatments.

The compounds of formula (1) are preferably used for agricultural, horticultural and turfgrass purposes in the form of a composition.

In order to apply a compound of formula (I) to a plant, to a seed of a plant, to the locus of the plant or seed or to soil or any other growth medium, a compound of formula (I) is usually formulated into a composition which includes, in addition to the compound of formula (I), a suitable inert diluent or carrier and, optionally, a surface active agent (SFA). SFAs are chemicals that are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of fungi such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides a fungicidal composition comprising a fungicidally effective amount of a compound of formula (I) and a suitable carrier or diluent therefor.

In a still further aspect the invention provides a method of combating and controlling fungi at a locus, which comprises treating the fungi, or the locus of the fungi with a fungicidally effective amount of a composition comprising a compound of formula (I). The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone), alcohols (such as benzyl alcohol, furfuryl alcohol or butanol), N-alkylpyrrolidones (such as N-methylpyrrolidone or N-octyl-pyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at ambient temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents that have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurised, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier). Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts. Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefin sulphonates, taurates and lignosulphonates. Suitable SFAs of the amphoteric type include betaines, propionates and glycinates. Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or aftapulgite).

A compound of formula (I) may be applied by any of the known means of applying fungicidal compounds. For example, it may be applied, formulated or unformulated, to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilisers (for example nitrogen-, potassium- or phosphorus-containing fertilisers). Suitable formulation types include granules of fertiliser. The mixtures suitably contain up to 25% by weight of the compound of formula (I).

The invention therefore also provides a fertiliser composition comprising a fertiliser and a compound of formula (I).

The compositions of this invention may contain other compounds having biological activity, for example micronutrients or compounds having similar or complementary fungicidal activity or which possess plant growth regulating, herbicidal, insecticidal, nematicidal or acaricidal activity.

By including another fungicide, the resulting composition may have a broader spectrum of activity or a greater level of intrinsic activity than the compound of formula (I) alone. Further the other fungicide may have a synergistic effect on the fungicidal activity of the compound of formula (I).

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I); or help to overcome or prevent the development of resistance to individual components. The particular additional active ingredient will depend upon the intended utility of the composition.

Examples of fungicidal compounds which may be included in the composition of the invention are AC 382042 (N-(1-cyano-1,2-dimethylpropyl)-2-(2,4-dichlorophenoxy)propionamide), acibenzolar-5-methyl, alanycarb, aldimorph, anilazine, azaconazole, azafenidin, azoxystrobin, benalaxyl, benomyl, benthiavalicarb, biloxazol, bitertanol, blasticidin S, boscalid (new name for nicobifen), bromuconazole, bupirimate, captafol, captan, carbendazim, carbendazim chlorhydrate, carboxin, carpropamid, carvone, CGA 41396, CGA 41397, chinomethionate, chlorbenzthiazone, chlorothalonil, chlorozolinate, clozylacon, copper containing compounds such as copper oxychloride, copper oxyquinolate, copper sulphate, copper tallate, and Bordeaux mixture, cyamidazosulfamid, cyazofamid (IKF-916), cyflufenamid, cymoxanil, cyproconazole, cyprodinil, debacarb, di-2-pyridyl disulphide 1,1′-dioxide, dichlofluanid, diclocymet, diclomezine, dicloran, diethofencarb, difenoconazole, difenzoquat, diflumetorim, O,O-di-isopropyl-S-benzyl thiophosphate, dimefluazole, dimetconazole, dimethirimol, dimethomorph, dimoxystrobin, diniconazole, dinocap, dithianon, dodecyl dimethyl ammonium chloride, dodemorph, dodine, doguadine, edifenphos, epoxiconazole, ethaboxam, ethirimol, ethyl (Z)-N-benzyl-N([methyl(methyl-thioethylideneaminooxycarbonyl)amino]thio)-β-alaninate, etridiazole, famoxadone, fenamidone, fenarimol, fenbuconazole, fenfuram, fenhexamid, fenoxanil (AC 382042), fenpiclonil, fenpropidin, fenpropimorph, fentin acetate, fentin hydroxide, ferbam, ferimzone, fluazinam, fludioxonil, flumetover, flumorph, fluoroimide, tluoxastrobin, fluquinconazole, flusilazole, flusulfamide, flutolanil, flutriafol, folpet, fosetyl-aluminium, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb, isopropanyl butyl carbamate, isoprothiolane, kasugamycin, kresoxim-methyl, LY186054, LY211795, LY 248908, mancozeb, maneb, mefenoxam, mepanipyrim, mepronil, metalaxyl, metalaxyl M, metconazole, metiram, metiram-zinc, metominostrobin, metrafenone, MON65500 (N-allyl-4,5-dimethyl-2-trimethylsilylthiophene-3-carboxamide), myclobutanil, NTN0301, neoasozin, nickel dimethyldithiocarbamate, nitrothale-isopropyl, nuarimol, ofurace, organomercury compounds, orysastrobin, oxadixyl, oxasulfuron, oxolinic acid, oxpoconazole, oxycarboxin, pefurazoate, penconazole, pencycuron, phenazin oxide, phosphorus acids, phthalide, picoxystrobin, polyoxin D, polyram, probenazole, prochloraz, procymidone, propamocarb, propamocarb hydrochloride, propiconazole, propineb, propionic acid, proquinazid, prothioconazole, pyraclostrobin, pyrazophos, pyrifenox, pyrimethanil, pyroquilon, pyroxyfur, pyrrolnitrin, quaternary ammonium compounds, quinomethionate, quinoxyfen, quintozene, silthiofam (MON 65500), S-imazalil, simeconazole, sipconazole, sodium pentachlorophenate, spiroxamine, streptomycin, sulphur, tebuconazole, tecloftalam, tecnazene, tetraconazole, thiabendazole, thifluzamide, 2-(thiocyanomethylthio)benzothiazole, thiophanate-methyl, thiram, tiadinil, timibenconazole, tolclofos-methyl, tolylfluanid, triadimefon, triadimenol, triazbutil, triazoxide, tricyclazole, tridemorph, trifloxystrobin, triflumizole, triforine, triticonazole, validamycin A, vapam, vinclozolin, XRD-563, zineb, ziram, zoxamide and the compounds of the formulae:

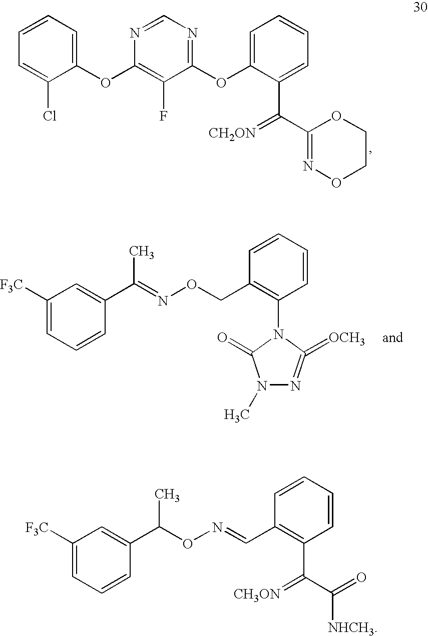

The compounds of formula (I) may be mixed with soil, peat or other rooting media for the protection of plants against seed-borne, soil-borne or foliar fungal diseases.

Some mixtures may comprise active ingredients, which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation. The invention is illustrated by the following Examples in which the following abbreviations are used:

ml = millilitres
g = grammes
THF = tetrahydrofuran
M⁺ = mass ion
s = singlet
d = doublet
HOBT = 1-hydroxybenzotriazole
bs = broad singlet
t = triplet
q = quartet
m = multiplet
ppm = parts per million
M = molar
m.p. = melting point (uncorrected)
b.p. = boiling point
DMSO = dimethylsulphoxide
DMF = N,N-dimethylformamide
HOAT = 7-aza-1-hydroxybenzotriazole
NMR = nuclear magnetic resonance
HPLC = high performance liquid chromatography
TLC = thin layer chromatography
glc = gas-liquid chromatography
EDC = 1-ethyl-3-N,N-dimethylamino propylcarbodiimide hydrochloride

### EXAMPLE 1

This examples illustrates the preparation of 2-(3-Bromo-7-chloro-quinolin-6-yloxy)-N-tert-butyl-2-methylsulfanyl-acetamide (Compound No. 12 of Table 2)

### Stage 1. Preparation of 3-bromo-7-chloro-quinolin-6-ol

### Step 1

3-Chloro-4-methoxy aniline (10 g) in acetic acid (100 ml) was treated with 2,2,3 tribromopropanal (18.8 g) and the mixture was stirred at room temperature for 2 h after which it was diluted with water and extracted with ethyl acetate. The organic phase was washed with 2N NaOH, dried over sodium sulphate, filtered and evaporated under reduced pressure to give the desired product, 3-bromo-7-chloro-6-methoxy-quinoline, as a yellow solid after chromatography (silica; hexane/ethyl acetate) (M+274). ¹H NMR (CDCl₃) δ ppm: 8.78 (1H, d); 8.21 (1H, d); 8.10 (1H, s); 7.03 (1H, s).

### Step 2

A mixture of the product from Step 1 (1.4 g) and hydrobromic acid (48 wt % solution in water) (100 ml×2) was refluxed for 62 hours. The mixture was cooled to ambient temperature, diluted with water, treated with sodium hydrogen carbonate and extracted with ethyl acetate. The extract was dried over magnesium sulfate, filtered and evaporated under reduced pressure to give the required product, 3-bromo-7-chloro-quinolin-6-ol (M+260). ¹H NMR (DMSO) δ ppm: 7.34 (1H, s); 7.07 (1H, s); 8.60 (1H, d); 8.73 (1H, d); 11.12 (1H, s).

### Stage 2: Preparation of (3-Bromo-7-chloro-quinolin-6-yloxy)-methylsulfanyl-acetic acid methyl ester

### Step 1

To a stirred solution of methyl (methylthio)acetate (10.8 ml) in dichloromethane (300 ml) cooled to −15° C. was added dropwise sulphuryl chloride (8.1 ml). The mixture was allowed to warm to room temperature over two hours and then concentrated under reduced pressure to give crude chloro-methylsulfanyl-acetic acid methyl ester as a colourless liquid. The product was used in the next step without further purification. ¹H NMR (CDCl₃) δ ppm: 2.33 (3H, s); 3.83 (3H, s); 5.49 (1H, s).

### Step 2

To a stirred solution of 3-bromo-7-chloro-quinolin-6-ol from Stage 1, Step 2 (1.1 g) in dry DMF (17 ml) containing anhydrous potassium carbonate (3.1 g) at ambient temperature was added chloro-methylsulfanyl-acetic acid methyl ester (0.79 g) dropwise. The mixture was heated for 1 hour at 60-65° C. then cooled to ambient temperature, diluted with water and extracted with ethyl acetate. The extracts were combined, washed with brine, dried over magnesium sulfate, filtered and evaporated under reduced pressure. Purification of the crude material by chromatography (silica; ethyl acetate/hexane 1:4 by volume) provided the title compound, 3-bromo-7-chloro-quinolin-6-yloxy)-methylsulfanyl-acetic acid methyl ester (M+378). ¹H NMR (CDCl₃) δ ppm: 2.29 (3H, s); 3.89 (3H, s); 5.74 (1H, s) 7.18 (1H, 8); 8.16 (1H, s); 8.23 (1H, s); 8.82 (1H, s).

### Stage 3: Preparation of (3-Bromo-7-chloro-quinolin-6-yloxy)-methylsulfanyl-acetic acid

To a stirred solution of the product from Stage 2, Step 2 (1.1 g) in ethanol (14 ml) at ambient temperature was added a solution of 2N sodium hydroxide in water (2.2 ml). The mixture was stirred at room temperature for 1 hour and then poured into ice-water and acidified with 2M hydrochloric acid. The resulting precipitate was filtered from solution, washed with cold water and dried in vacuo to give (3-bromo-7-chloro-quinolin-6-yloxy)-methylsulfanyl-acetic acid (M+364). ¹H NMR (DMSO-d₆) δ ppm: 2.20 (3H, s); 6.18 (1H, s) 7.57 (1H, s); 8.19 (1H, s); 8.28 (1H, d); 8.88 (1H, d); 13.7 (1H, bs).

### Stage 4: Preparation of 2-(3-Bromo-7-chloro-quinolin-6-yloxy)-N-tert-butyl-2-methylsulfanyl-acetamide

The product from Stage 3 above, 3-bromo-7-chloro-quinolin-6-yloxy)-methylsulfanyl-acetic acid (85 mg) from in dry N,N-dimethylformamide (2 ml) was treated with t-butylamine (17 mg), N-(3-dimethylamino-propyl)-N′-ethyl carbodiimide hydrochloride (45 mg), HOAT (32 mg) and triethylamine (24 mg) at ambient temperature with stirring for 3 hours. The mixture was poured into water, extracted with ethyl acetate (three times) and the extracts combined, washed with saturated aqueous sodium carbonate solution, water (three times) then dried over magnesium sulfate, filtered and evaporated under reduced pressure to give an oil. The oil was fractionated by chromatography (silica; hexane/ethyl acetate, 3:1 by volume) to give the required product as a white solid (m.p. 172-174° C., M+419). ¹H NMR (CDCl₃) δ ppm: 1.46 (9H, s); 2.18 (3H, s); 5.63 (1H, s); 6.89 (1H, bs); 7.28 (1H, s); 7.28 (1H, s); 8.18 (1H, s), 8.27 (1H, d); 8.83 (1H, d).

The following amides were prepared using a similar procedure.

Compound No. 238 of Table 2: using 1,1-dimethyl-prop-2-ynylamine, m.p. 169-171° C.; ¹H NMR (CDCl₃) δ ppm: 1.75 (6H, s); 2.19 (3H, s); 2.41 (1H, s); 5.69 (1H, s); 7.16 (1H, bs); 7.29 (1H, s); 8.18 (1H, s); 8.27 (1H, d); 8.83 (1H, d).

Compound No. 52 of Table 2: using 2-amino-3-methoxy-2-methyl-propionitrile, ¹H NMR (CDCl₃) δ ppm: diastereoisomeric mixture (1/1); 1.82 and 1.84 (3H, 2×s); 2.18 and 2.20 (3H, 2×s); 3.52 and 3.53 (3H, 2×s); 3.64-3.84 (2H, 2×dd); 5.76 and 5.77 (1H, 2×s); 7.29 and 7.30 (1H, 2×s); 7.59 and 7.61 (1H, 2×bs); 8.18 (1H, s); 8.29 (1H, d); 8.85 (1H, s).

Compound No. 244 of Table 2: using 1,1-dimethyl-but-2-ynylamine, m.p. 160-162° C.; ¹H NMR (CDCl₃) δ ppm: 1.71 (6H, s); 1.83 (3H, s); 2.19 (3H, s); 5.66 (1H, s); 7.15 (1H, bs); 7.27 (1H, s); 8.18 (1H, s); 8.27 (1H, d); 8.83 (1H, d).

Compound No. 251 of Table 2: using 4-methoxy-1,1-dimethyl-but-2-ynylamine, m.p. 124-125° C.; ¹H NMR (CDCl₃) δ ppm: 1.75 (6H, s); 2.19 (3H, s); 3.39 (3H, s); 4.13 (2H, s); 5.67 (1H, s); 7.17 (1H, bs); 7.28 (1H, s); 8.18 (1H, s); 8.28 (1H, d); 8.84 (1H, d).

### EXAMPLE 2

This examples illustrates the preparation of 2-(3-Bromo-7-methyl-quinolin-6-yloxy)-N-tert-butyl-2-methylsulfanyl-acetamide (Compound No. 12 of Table 13)

### Stage 1. Preparation of 3-bromo-7-methyl-quinolin-6-ol

4-Amino-2-methyl-phenol (5 g) in acetic acid (60 ml) was treated with 2,2,3 tribromopropanal (11.9 g) and the mixture was stirred at room temperature for 3 h after which it was diluted with water and extracted with ethyl acetate. The organic phase was washed with aqueous NH₄OH, dried over sodium sulphate, filtered and evaporated under reduced pressure to give the required product, 3-bromo-7-methyl-6-quinolin-6-ol which was used as such in the following step (M+240), ¹H NMR (DMSO-d6) δ ppm: 2.32 (3H, s); 7.12 (1H, s); 7.75 (1H, s); 8.46 (1H, d); 8.64 (1H, d); 10.35 (1H, bs).

### Stage 2: Preparation of methyl 3-bromo-7-methyl-quinolinyl-6-oxy-2-methylthioacetate

In a similar procedure to Stage 2, Step 2 of Example 1, 3-bromo-7-methyl-quinolin-6-ol was reacted with methyl 2-bromo-2-methylthioacetate to give methyl 3-bromo-7-methyl-quinolinyl-6-oxy-2-methylthio-acetate (M+358). ¹H NMR (CDCl₃) δ ppm: 2.25 (3H, s); 2.50 (3H, s); 3.88 (3H, s); 5.73 (1H, s); 6.99 (1H, s); 7.86 (1H, s); 8.19 (1H, d), 8.76 (1H, d).

### Stage 3: Preparation of (3-Bromo-7-methyl-quinolin-6-yloxy)-methylsulfanyl-acetic acid

In a similar procedure to Stage 3 of Example 1, methyl 3-bromo-7-methyl-quinolinyl-6-oxy-2-methylthio-acetate was hydrolysed to give (3-bromo-7-methyl-quinolin-6-yloxy)-methylsulfanyl-acetic acid (M+344). ¹H NMR (DMSO) δ ppm: 2.19 (3H, s); 2.42 (3H, s); 6.07 (1H, s); 7.42 (1H, s); 7.86 (1H, s); 8.52 (1H, s), 8.78 (1H, s), 13.55 (1H, bs).

### Stage 4: Preparation of 2-(3-Bromo-7-methyl-quinolin-6-yloxy)-N-tert-butyl-2-methylsulfanyl-acetamide

In a similar procedure to Stage 4 of Example 1,2-(3-bromo-7-methyl-quinolinyl-6-oxy)-2-methylthioacetic acid was condensed with t-butylamine to give 2-(3-bromo-7-methyl-quinolin-6-yloxy)-N-tert-butyl-2-methylsulfanyl-acetamide (m.p. 132-134° C., M+344). ¹H NMR (CDCl₃) δ ppm: 1.44 (9H, s); 2.19 (3H, s); 2.49 (3H, s); 5.60 (1H, s); 6.48 (1H, bs); 7.10 (1H, s); 7.89 (1H, s); 8.21 (1H, d), 8.78 (1H, d).

The following amides were prepared using a similar procedure.

Compound No. 16 of Table 13: using 1,1-dimethyl-propylamine, m.p. 135-137° C.; ¹H NMR (CDCl₃) δ ppm: 0.88 (3H, t); 1.39 (6H, s); 1.79 (2H, q); 2.20 (3H, s); 2.48 (3H, s); 5.59 (1H, s); 6.39 (1H, bs); 7.10 (1H, s); 7.89 (1H, s); 8.21 (1H, d); 8.78 (1H, d).

Compound No. 238 of Table 13: using 1,1-dimethyl-prop-2-ynylamine, m.p. 141-146° C.; ¹H NMR (CDCl₃) δ ppm: 1.74 (6H, s); 2.19 (3H, s); 2.41 (1H, s); 2.50 (3H, s); 5.65 (1H, s); 6.77 (1H, bs); 7.10 (1H, s); 7.89 (1H, s); 8.21 (1H, d); 8.78 (1H, d).

Compound No. 52 of Table 13: using 2-amino-3-methoxy-2-methyl-propionitrile, ¹H NMR (CDCl₃) δ ppm: diastereoisomeric mixture (1/1); 1.81 and 1.83 (3H, 2×s); 2.20 and 2.21 (3H, 2×s); 2.48 (3H, s); 3.50 and 3.53 (3H, 2×s); 3.61-3.82 (2H, 2×dd); 5.72 and 5.75 (1H, 2×s); 7.11 and 7.13 (1H, 2×s); 7.32 and 7.34 (1H, 2×bs); 7.90 (1H, s); 8.23 (1H, s); 8.79 (1H, s).

Compound No. 221 of Table 13: using 1,1-dimethyl-2-prop-2-ynyloxy-ethylamine, m.p. 114-116° C.; ¹H NMR (CDCl₃) δ ppm: 1.41 (3H, s); 1.44 (3H, s); 2.18 (3H, s); 2.50 (1H, t); 3.54 (2H, dd); 4.19 (2H, d); 5.61 (1H, s); 6.92 (1H, bs); 7.10 (1H, s); 7.88 (1H, s); 8.22 (1H, d); 8.77 (1H, d).

Compound No. 251 of Table 13: using 4-methoxy-1,1-dimethyl-but-2-ynylamine, m.p. 129-132° C.; ¹H NMR (CDCl₃) δ ppm: 1.73 (6H, s); 2.20 (3H, s); 2.49 (3H, s); 3.37 (3H, s); 4.11 (2H, s); 5.63 (1H, s); 6.76 (1H, bs); 7.10 (1H, s); 7.89 (1H, s); 8.21 (1H, d); 8.78 (1H, d).

### EXAMPLE 3

This examples illustrates the preparation of 2-(3-Bromo-7-fluoro-quinolin-6-yloxy)-N-tert-butyl-2-methylsulfanyl-acetamide (Compound No. 12 of Table 6)

### Stage 1. Preparation of 3-bromo-7-fluoro-quinolin-6-ol

### Step 1

In a similar procedure to Step 1, Stage 1 of Example 1, 3-fluoro-4-methoxy-phenylamine (2 g) in acetic acid (25 ml) was treated with 2,2,3 tribromopropanal (4.2 g) to give 3-bromo-7-fluoro-6-methoxy-quinoline (M+258). ¹H NMR (CDCl₃) δ ppm: 4.02 (3H, s); 7.07 (1H, d); 7.75 (1H, d); 8.23 (1H, d); 8.78 (1H, d).

### Step 2

In a similar procedure to Step 1, Stage 2 of Example 1, 3-bromo-7-fluoro-6-methoxy-quinoline from the previous step was treated with hydrobromic acid to give 3-bromo-7-fluoro-quinolin-6-ol (M+244). ¹H NMR (DMSO-d6) δ ppm: 7.35 (1H, d); 7.77 (1H, d); 8.59 (1H, d); 8.74 (1H, d), 10.93 (1H, s).

### Stage 2: Preparation of (3-bromo-7-fluoro-quinolin-6-yloxy)-methylsulfanyl-acetic acid methyl ester

In a similar procedure to Stage 2, Step 2 of Example 1, 3-bromo-7-fluoro-quinolin-6-ol from the previous step was reacted with methyl 2-bromo-2-methylthioacetate from Stage 2, Step 1 of Example 1, to give methyl 3-bromo-7-fluoro-quinolinyl-6-oxy-2-methylthioacetate (M+362). ¹H NMR (CDCl₃) δ ppm: 2.27 (3H, s); 3.89 (3H, s); 5.77 (1H, s) 7.26 (1H, d); 7.77 (1H, s); 8.23 (1H, s); 8.83 (1H, s).

### Stage 3: Preparation of (3-bromo-7-fluoro-quinolin-6-yloxy)-methylsulfanyl-acetic acid

In a similar procedure to Stage 3 of Example 1, methyl 3-bromo-7-fluoro-quinolinyl-6-oxy-2-methylthio-acetate was hydrolysed to give (3-bromo-7-fluoro-quinolin-6-yloxy)-methylsulfanyl-acetic acid (M⁺ 348). ¹H NMR (DMSO-d6) δ ppm: 6.16 (1H, s); 7.71 (1H, d) 7.90 (1H, d); 8.62 (1H, d), 8.88 (1H, d), 13.69 (1H, bs).

### Stage 4: Preparation of 2-(3-bromo-7-fluoro-quinolin-6-yloxy)-N-tert-butyl-2-methylsulfanyl-acetamide

In a similar procedure to Stage 4 of Example 1,2-(3-bromo-7-fluoro-quinolinyl-6-oxy)-2-methylthioacetic acid was condensed with t-butylamine to give 2-(3-bromo-7-fluoro-quinolin-6-yloxy)-N-tert-butyl-2-methylsulfanyl-acetamide (m.p. 144-145° C., M⁺ 403). ¹H NMR (CDCl₃) δ ppm: 1.45 (9H, s); 2.19 (3H, s); 5.62 (1H, s); 6.62 (1H, bs); 7.30 (1H, d); 7.79 (1H, d); 8.26 (1H, d), 8.84 (1H, d).

The following amides were prepared using a similar procedure.

Compound No. 238 of Table 6: using 1,1-dimethyl-prop-2-ynylamine, m.p. 167-169° C.; ¹H NMR (CDCl₃) δ ppm: 1.75 (6H, s); 2.21 (3H, s); 2.41 (1H, s); 2.50 (3H, s); 5.68 (1H, s); 6.89 (1H, bs); 7.33 (1H, d); 7.79 (1H, d); 8.26 (1H, d); 8.84 (1H, d).

Compound No. 52 of Table 6: using 2-amino-3-methoxy-2-methyl-propionitrile, ¹H NMR is (CDCl₃) δ ppm: diastereoisomeric mixture (1/1); 1.81 and 1.83 (3H, 2×s); 2.21 and 2.22 (3H, 2×s); 3.52 and 3.54 (3H, 2×s); 3.64-3.84 (2H, 2×dd); 5.75 and 5.76 (1H, 2×s); 7.33-7.36 (2H, m); 7.80 (1H, d); 8.28 (1H, d); 8.85 (1H, d).

Compound No. 221 of Table 6: using 1,1-Dimethyl-2-prop-2-ynyloxy-ethylamine, m.p. 115-117° C.; ¹H NMR (CDCl₃) δ ppm: 1.44 (3H, s); 1.46 (3H, s); 2.20 (3H, s); 2.45 (1H, t); 3.61 (2H, dd); 4.20 (2H, d); 5.63 (1H, s); 6.90 (1H, bs); 7.28 (1H, d); 7.78 (1H, d); 8.26 (1H, d); 8.83 (1H, d).

Compound No. 251 of Table 6: using 4-methoxy-1,1-dimethyl-but-2-ynylamine, m.p. 115-117° C.; ¹H NMR (CDCl₃) δ ppm: 1.74 (6H, s); 2.21 (3H, s); 2.49 (3H, s); 3.38 (3H, s); 4.12 (2H, s); 5.65 (1H, s); 6.88 (1H, bs); 7.31 (1H, d); 7.79 (1H, d); 8.26 (1H, d); 8.84 (1H, d).

Compound No. 244 of Table 6: using 1,1-dimethyl-but-2-ynylamine, m.p. 130-132° C.; ¹H NMR (CDCl₃) δ ppm: 1.70 (6H, s); 1.83 (3H, s); 2.21 (3H, s); 5.65 (1H, s); 6.86 (1H, bs); 7.31 (1H, d); 7.78 (1H, d); 8.25 (1H, d); 8.84 (1H, d).

Compound No. 68 of Table 6: using 2-ethoxy-1,1-dimethyl-ethylamine, m.p. 111-113° C.; ¹H NMR (CDCl₃) δ ppm: 1.21 (3H, t); 1.43 (H, s); 1.46 (3H, s); 2.20 (3H, s); 3.43 (2H, dd); 3.51-3.58 (2H, m), 5.63 (1H, s); 7.17 (1H, bs); 7.28 (1H, d); 7.78 (1H, d); 8.25 (1H, d); 8.83 (1H, d).

Compound No. 277 of Table 6: using 5-methoxy-1,1-dimethyl-pent-2-ynylamine, m.p. 104-106° C.; ¹H NMR (CDCl₃) δ ppm: 1.71 (6H, s); 2.20 (3H, s); 2.48 (2H, t); 3.37 (3H, s); 3.50 (2H, t); (5.65 (1H, s); 6.88 (1H, bs); 7.31 (1H, d); 7.79 (1H, d); 8.26 (1H, d); 8.84 (1H, d).

Compound No. 256 of Table 6: using 6-chloro-1,1-dimethyl-hex-2-ynylamine, m.p. 95-97° C.; ¹H NMR (CDCl₃) δ ppm: 1.70 (6H, s); 1.96 (2H, quin), 2.20 (3H, s); 2.90 (2H, t); 3.68 (2H, t); 3.50 (2H, t); 5.66 (1H, s); 6.87 (1H, bs); 7.31 (1H, d); 7.79 (1H, d); 8.26 (1H, d); 8.84 (1H, d).

### EXAMPLE 4

This examples illustrates the preparation of 2-(3-iodo-7-fluoro-quinolin-6-yloxy)-N-tert-butyl-2-methylsulfanyl-acetamide (Compound No. 12 of Table 7)

### Stage 1: Preparation of 3-iodo-6-hydroxyquinoline

To a stirred mixture of 3-bromo-7-fluoro-6-hydroxyquinoline from Example 3, Stage 1, Step 2 (0.50 g), sodium iodide (0.62 g) and copper iodide (0.08 g) in dioxane (3.0 ml) was added N,N,N′,N′-tetramethyl-ethane-1,2-diamine (0.07 g) in a sealed tube. The mixture was stirred at 120° C. for 14 h and upon cooling was treated with aqueous ammonia followed by aqueous hydrochloric acid. Extraction with ethyl acetate, drying of the organic phase over magnesium sulphate, filtration and evaporation under reduced pressure gave the required product (M+290) as a light brown coloured powder that was used as such in the next step. ¹H NMR (CDCl₃) δ ppm: 7.30 (1H, d); 7.72 (1H, d); 8.02 (1H, d), 8.73 (1H, d); 8.83 (1H, d), 10.88 (1H, s).

### Stage 2: Preparation of N-tert-Butyl-2-(3-iodo-7-fluoro-quinolin-6-yloxy)-2-methylsulfanyl-acetamide

In a similar procedure to Stage 2, Step 1 of Example 1, 3-iodo-7-fluoro-6-hydroxy-quinoline was reacted with chloromethylsulfanyl-acetic acid ethyl ester to give (3-iodo-7-fluoro-quinolin-6-yloxy)-methylsulfanyl-acetic acid ethyl ester (M+408).

In a similar procedure to Stage 3 of Example 1, (3-iodo-7-fluoro-quinolin-6-yloxy)-methylsulfanyl-acetic acid ethyl ester was hydrolysed to give (3-iodo-7-fluoro-quinolin-6-yloxy)-methylsulfanyl-acetic acid (M+394).

In a similar procedure to Stage 4 of Example 1, (3-iodo-7-fluoro-quinolin-6-yloxy)-methylsulfanyl-acetic acid was condensed with t-butylamine to give N-tert-butyl-2-(3-iodo-quinolin-6-yloxy)-2-methylsulfanyl-acetamide as a white solid (m.p. 165-166° C., M⁺ 449).

¹H NMR (CDCl₃) δ ppm: 1.44 (9H, s); 2.18 (3H, s); 5.60 (1H, s); 6.62 (1H, bs); 7.27 (1H, d); 7.76 (1H, dd); 8.47 (1H, d); 8.97 (1H, d).

The following amides were prepared using a similar procedure.

Compound No. 238 of Table 7: using 1,1-dimethyl-prop-2-ynylamine, m.p. 150-152° C.; ¹H NMR (CDCl₃) δ ppm: 1.75 (6H, s); 2.20 (3H, s); 2.40 (1H, s); 5.66 (1H, s); 6.88 (1H, bs); 7.27 (1H, s); 7.77 (1H, s); 8.47 (1H, d); 8.97 (1H, d).

Compound No. 52 of Table 7: using 2-amino-3-methoxy-2-methyl-propionitrile, ¹H NMR (CDCl₃) δ ppm: diastereoisomeric mixture (1/1); 1.82 and 1.84 (3H, 2×s); 2.20 and 2.22 (3H, 2×s); 3.53 and 3.54 (3H, 2×s); 3.64-3.84 (2H, 2×dd); 5.73 and 5.75 (1H, 2×s); 7.27-7.36 (3H, m); 7.77 (1H, d); 8.49 (1H, d); 8.98 (1H, s).

Compound No. 244 of Table 7: using 1,1-dimethyl-but-2-ynylamine, m.p. 142-145° C.; ¹H NMR (CDCl₃) δ ppm: 1.70 (6H, s); 1.83 (3H, s); 2.20 (3H, s); 5.64 (1H, s); 6.87 (1H, bs); 7.27 (1H, d); 7.76 (1H, d); 8.47 (1H, d); 8.96 (1H, d).

Compound No. 251 of Table 7: using 4-methoxy-1,1-dimethyl-but-2-ynylamine, m.p. 110-112° C.; ¹H NMR (CDCl₃) δ ppm: 1.74 (6H, s); 2.20 (3H, s); 3.38 (3H, s); 4.13 (2H, s); 5.65 (1H, s); 6.89 (1H, bs); 7.27 (1H, d); 7.78 (1H, d); 8.47 (1H, d); 8.97 (1H, d).

### EXAMPLE 5

This examples illustrates the preparation of N-tert-butyl-2-(7-fluoro-3-thiophen-3-yl-quinolin-6-yloxy)-2-methylsulfanyl-acetamide (Compound No. 12 of Table 30).

Stage 1: A stirred mixture of 2-(3-bromo-7-fluoro-quinolin-6-yloxy)-N-tert-butyl-2-methylsulfanyl-acetamide from Example 3, Stage 4 (160 mg), bis(triphenylphosphine)palladium (II) chloride (1.4 mg), 3-thiophene boronic acid (56 mg) and sodium hydrogen carbonate (101 mg)) in dioxane/water (1:1 mixture, 4.8 ml) was heated to 70° C. for 14 h. Upon cooling, the reaction mixture was treated with saturated aqueous sodium hydrogen carbonate (5 ml). Extraction with ethyl acetate, drying of the organic phase over magnesium sulphate, filtration and evaporation under reduced pressure gave the crude product which was fractionated by chromatography (silica; hexane/ethyl acetate, 2:1 by volume) to give the required product as a white solid (m.p. 170-173° C., M+405). ¹H NMR (CDCl₃) δ ppm: 1.46 (9H, s); 2.20 (3H, s); 5.64 (1H, s); 6.66 (1H, bs); 7.42 (1H, d); 7.51 (2H, m); 7.66 (1H, m), 7.80 (1H, d), 8.22 (1H, d); 9.13 (1H, d).

The following amide was prepared using a similar procedure. 2-(7-Fluoro-3-thiophen-3-yl-quinolin-6-yloxy)-N-(4-methoxy-1,1-dimethyl-but-2-ynyl)-2-methyl-sulfanyl-acetamide (Compound No. 251 of Table 30) m.p. 132-134° C.; ¹H NMR (CDCl₃) δ ppm: 1.75 (6H, s); 2.22 (3H, s); 3.39 (3H, s); 4.13 (2H, s); 5.68 (1H, s); 6.93 (1H, bs); 7.42 (1H, d); 7.51 (2H, m); 7.66 (1H, m), 7.82 (1H, d), 8.22 (1H, d); 9.13 (1H, d). prepared from 2-(3-Bromo-7-fluoro-quinolin-6-yloxy)-N-(4-methoxy-1,1-dimethyl-but-2-ynyl)-2-methylsulfanyl-acetamide from Example 3, stage 4, above.

### EXAMPLE 6

This Example illustrates the fungicidal properties of compounds of formula (I).

The compounds were tested in a leaf disk assay, with methods described below. The test compounds were dissolved in DMSO and diluted into water to 200 ppm. In the case of the test on Pythium ultimum, they were dissolved in DMSO and diluted into water to 20 ppm.

Erysiphe graminis f.sp. tritici (wheat powdery mildew): Wheat leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Puccinia recondita f.sp. tritici (wheat brown rust): Wheat leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed nine days after inoculation as preventive fungicidal activity.

Septoria nodorum (wheat glume blotch): Wheat leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Pyrenophora teres (barley net blotch): Barley leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Pyricularia oryzae (rice blast): Rice leaf segments were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Botrytis cinerea (grey mould): Bean leaf disks were placed on agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Phytophthora infestans (late blight of potato on tomato): Tomato leaf disks were placed on water agar in a 24-well plate and sprayed with a solution of the test compound. After allowing to dry completely, for between 12 and 24 hours, the leaf disks were inoculated with a spore suspension of the fungus. After appropriate incubation the activity of a compound was assessed four days after inoculation as preventive fungicidal activity.

Plasmopara viticola (downy mildew of grapevine): Grapevine leaf disks were placed on agar in a 24-well plate and sprayed a solution of the test compound. After allowing to dry completely, for between 12 and 24 hours, the leaf disks were inoculated with a spore suspension of the fungus. After appropriate incubation the activity of a compound was assessed seven days after inoculation as preventive fungicidal activity.

Septoria tritici (leaf blotch): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24 C and the inhibition of growth was determined photometrically after 72 hrs.

Fusarium culmorum (root rot): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24 C and the inhibition of growth was determined photometrically after 48 hrs.

Pythium ultimum (Damping off): Mycelial fragments of the fungus, prepared from a fresh liquid culture, were mixed into potato dextrose broth. A solution of the test compound in dimethyl sulphoxide was diluted with water to 20 ppm then placed into a 96-well microtiter plate and the nutrient broth containing the fungal spores was added. The test plate was incubated at 24° C. and the inhibition of growth was determined photometrically after 48 hours.

The following compounds (number of compound first, followed by table number in brackets) gave at least 60% control of the following fungal infection at 200 ppm: Plasmopara viticola, compounds 9 (6), 12 (7), 38 (6), 52 (6), 52 (7), 60 (6), 95 (6), 221 (6), 221 (13), 235 (6), 238 (7), 238 (10), 244 (6), 244 (7), 251 (2), 251 (7), 251 (10), 251 (13), 251 (30), 256 (6), 264 (6), 277 (6), 278 (6), Phytophthora infestans, compounds 12 (6), 12 (10), 52 (7), 52 (13), 221 (6), 221 (13), 238 (7), 238 (10), 238 (13), 244 (6), 244 (7), 251 (7), 251 (10), 256 (6), 275 (7), 277 (6), 278 (6), Erysiphe graminis f.sp. tritici, compounds 9 (6), 12 (6), 12 (13), 28 (6), 38 (6), 52 (6), 52 (7), 52 (13), 68 (6), 86 (6), 181 (6), 189 (6), 221 (6), 221 (13), 235 (6), 238 (6), 238 (7), 238 (10), 238 (13), 244 (6), 251 (6), 251 (7), 251 (13), 251 (30), 256 (6), 264 (6), 268 (6), 275 (6), 275 (7), 277 (6), 278 (6), 281 (6), 284 (6), Pyricularia oryzae, compounds 9 (6), 52 (6), 52 (7), 68 (6), 221 (6), 238 (6), 244 (7), 251 (6), 251 (7), 275 (7), 277 (6), 278 (6), Puccinia recondita f.sp. tritici, compounds 52 (6), 52 (7), 68 (6), 221 (6), 238 (7), 251(2), 264 (6), Septoria nodorum, compounds 9 (6), 12 (2), 12 (7), 12 (10), 12 (13), 12 (30), 52 (6), 52 (7), 68 (6), 221 (6), 235 (6), 238 (6), 238 (7), 244 (6), 244 (7), 244 (13), 251 (6), 251 (7), 251 (13), 256 (6), 264 (6), 277 (6), 278 (6), 281 (6), Septoria tritici, compounds 9 (6), 12 (6), 12 (7), 12 (13), 16 (13), 38 (6), 52 (2), 52 (6), 52 (7), 52(13), 68 (6), 95 (6), 122 (6), 221 (6), 221 (13), 224 (6), 235 (6), 238 (6), 238 (7), 238 (13), 244 (2), 244 (6), 244 (7), 251 (6), 251 (7), 251 (13), 251 (2), 251 (30), 256 (6), 264 (6), 268 (6), 275 (7), 277 (6), 278 (6), 281 (6), 284 (6), 287 (6), 291 (6), Fusarium culmorum, compounds 9 (6), 12 (6), 12 (7), 221 (6), 238 (6), 238 (7), 244 (6), 244 (7), 251 (6), 251 (7), 251 (13), 284 (6), The following compounds (number of compound first, followed by table number in brackets) gave at least 60% control of the following fungal infection at 20 ppm: Pythium ultimum, compounds 9 (6), 12 (10), 52 (6), 52 (13), 221 (6), 221 (13), 235 (6), 238 (6), 244 (6), 251 (6), 251 (10), 251 (13), 251 (30), 264 (6), 275 (7), 278 (6), 281 (6).

1. A compound of the general formula I 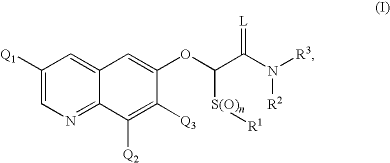
wherein
Q¹, Q² and Q³, independently of each other, are halogen, cyano, nitro, azido, optionally substituted C₁₋₆ alkyl, optionally substituted C₃₋₆ cycloalkyl, optionally substituted C₃₋₆-heterocyclyl, which contains at least one heteroatom selected from sulphur, oxygen or NR⁰, where R⁰ is hydrogen or optionally substituted C₁₋₆alkyl, optionally substituted C₃₋₆ cycloalkyl(C₁₋₄)alkyl, optionally substituted C₂₋₆ alkenyl, optionally substituted C₂₋₆ alkynyl, optionally substituted C₁₋₆ alkoxy, optionally substituted C₂₋₆ alkenyloxy, optionally substituted C₂₋₆ alkynyloxy, optionally substituted aryl, optionally substituted aryloxy, optionally substituted aryl(C₁₋₆)alkyl, optionally substituted aryl(C₁₋₆)alkoxy, optionally substituted heteroaryl, optionally substituted heteroaryloxy, optionally substituted heteroaryl(C₁₋₆)alkyl, optionally substituted heteroaryl(C₁₋₆)alkoxy, —SF₅ or —S(O)ᵤ(C₁₋₆)alkyl, wherein u is 0, 1 or 2 and the alkyl group is optionally substituted with halogen, or
Q¹, Q² and Q³, independently of each other, are —OSO₂(C₁₋₄)alkyl, wherein the alkyl group is optionally substituted with halogen, or
Q¹, Q² and Q³, independently of each other, are —CONR^{u}R^{v}, —COR^{u}, —CO₂R^{u}, —CR^{u}═NR^{v}, —NR^{u}R^{v}, —NR^{u}COR^{v}, —NR^{u}CO₂R^{v}, —SO₂NR^{u}R^{v} or —NR^{u}SO₂R^{w}, wherein R^{w} is optionally substituted C₁₋₆ alkyl and R^{u} and R^{v}, independently of each other, are hydrogen or C₁₋₆ alkyl optionally substituted with halogen, or, in the case of —CONR^{u}R^{v} or —SO₂NR^{u}R^{v}, R^{u}UR^{v} may join to form a 5- or 6-membered carbocyclic or heterocyclic ring containing a heteroatom selected from sulfur, oxygen and NR⁰, wherein R^{o} is hydrogen or optionally substituted C₁₋₆alkyl, or, in the case of —CR^{u}═NR^{v}, R^{v} is hydroxy or optionally substituted C₁₋₆alkoxy, optionally substituted aryloxy or optionally substituted heteroaryloxy, or
Q¹ and Q², independently of each other, additionally denote hydrogen,
R¹ is optionally substituted C₁₋₄ alkyl, optionally substituted C₂₋₄ alkenyl, optionally substituted C₂₋₄ alkynyl or optionally substituted C₃₋₄ cycloalkyl,
R² is hydrogen, C₁₋₈ alkyl, C₃₋₄ cycloalkyl, C₂₋₈ alkenyl, cyano, hydroxy, alkoxy, cyano(C₁₋₄)alkyl, C₁₋₄ alkoxy(C₁₋₄)alkyl, C₁₋₄ alkoxy(C₁₋₄)alkoxy(C₁₋₄)alkyl or benzyloxy(C₁₋₄)alkyl, wherein the phenyl ring is optionally substituted with C₁₋₄ alkoxy,
R³ is —(CR^{a}R^{b})ₚ(CR^{c}R^{d})_{q}(X)ᵣ(CR^{e}R^{f})ₛR⁴, wherein
R^{a}, R^{b}, R^{c}, R^{d}, R^{e} and R^{f}, independently of each other, are hydrogen, C₁₋₄ alkyl, halogen, cyano, hydroxy, C₁₋₄ alkoxy or C₁₋₄ alkoxycarbonyl, or
R^{a}R^{b}, R^{c}R^{d} or R^{e}R^{f} may join to form a 3 to 8 membered carbocyclic or heterocyclic ring containing a heteroatom selected from sulfur, oxygen and NR^{o}, wherein R^{o} is hydrogen or optionally substituted C₁₋₆alkyl,
X is (CO), (CO)O, O(CO), O, S(O)ₜ, wherein t is 0, 1 or 2, or X is NH or N(C₁₋₆)alkyl,
p, r and s, independently of each other, are 0 or 1,
q is 0, 1 or 2,
R⁴ is optionally substituted C₁₋₆ alkyl, optionally substituted C₂₋₆ alkenyl, —CR^{uu}═NR^{vv}, wherein R^{uu} is hydrogen or C₁₋₆ alkyl and R^{vv} is hydroxy or optionally substituted C₁₋₆alkoxy, optionally substituted aryloxy or optionally substituted heteroaryloxy, or —CH₂—C≡C—R⁵, wherein
R⁵ is hydrogen, C₁₋₈ alkyl optionally substituted with halogen, hydroxy, C₁₋₆ alkoxy, C₁₋₃ alkoxy(C₁₋₃)alkoxy, cyano, C₁₋₄ alkylcarbonyloxy, aminocarbonyloxy, mono- or di(C₁₋₄)-alkylaminocarbonyloxy, tri(C₁₋₄)alkylsilyloxy or —S(O)_{g}(C₁₋₆)alkyl, wherein g is 0, 1 or 2, or
R⁵ is C₃₋₆ cycloalkyl optionally substituted with halogen, hydroxy, C₁₋₆ alkoxy, C₁₋₃ alkoxy-(C₁₋₃)alkoxy, cyano, C₁₋₄ alkylcarbonyloxy, aminocarbonyloxy, mono- or di(C₁₋₄)alkyl-aminocarbonyloxy, tri(C₁₋₄)alkylsilyloxy or —S(O)_{g}(C₁₋₆)alkyl, wherein g is 0, 1 or 2, or
R⁵ is C₃₋₆ cycloalkyl(C₁₋₄)alkyl, wherein the alkyl and/or cycloalkyl moiety is optionally substituted with halogen, hydroxy, C₁₋₆ alkoxy, C₁₋₃ alkoxy(C₁₋₃)alkoxy, cyano, C₁₋₄ alkyl-carbonyloxy, aminocarbonyloxy, mono- or di(C₁₋₄)alkylaminocarbonyloxy, tri(C₁₋₄)alkyl-silyloxy or —S(O)_{g}(C₁₋₆)alkyl, wherein g is 0, 1 or 2, or
R⁵ is optionally substituted aryl, optionally substituted aryl(C₁₋₄)alkyl, optionally substituted aryloxy(C₁₋₄)alkyl, optionally substituted heteroaryl or optionally substituted heteroaryl(C₁₋₄)alkyl or optionally substituted heteroaryloxy(C₁₋₄)alkyl, or
R⁴ is optionally substituted C₃₋₆ cycloalkyl, optionally substituted C₅₋₆ cycloalkenyl, optionally substituted aryl, optionally substituted heteroaryl or an optionally substituted 5- to 8-membered ring optionally containing a heteroatom selected from sulfur, oxygen or NR⁰, wherein R^{o} is hydrogen or optionally substituted C₁₋₆alkyl, or
when R³ is —(CR^{a}R^{b})ₚ(CR^{c}R^{d})_{q}(X)ᵣ(CR^{e}R^{f})ₛR⁴, R² and R³ may join to form a 5- or 6-membered ring optionally substituted with halogen, C₁₋₄ alkyl, mono- or di-(C₁₋₄)alkylaminocarbonyl, and optionally containing a heteroatom selected from sulphur, oxygen and NR⁰⁰, wherein R⁰⁰ is C₁₋₄ alkyl optionally substituted with halogen, C₁₋₆ alkoxy or cyano, or R⁰⁰ is phenyl optionally substituted with nitro, C₁₋₄ alkyl, halo(C₁₋₄)-alkyl, C₁₋₄ alkylcarbonyl or heteroaryl, or R² and R³ may join to form an optionally substituted 6,6-membered bicycle,
R³ is —(CR³⁰R⁴⁰)C≡CR⁵⁰, wherein
R³⁰ and R⁴⁰, independently of each other, are hydrogen, C₁₋₆ alkyl, halo(C₁₋₄)alkyl, C₁₋₄ alkoxy(C₁₋₃)alkyl, C₂₋₃ alkenyl or C₂₋₃alkynyl, or
R³⁰ and R⁴⁰ join with the carbon atom to which they are attached to form a 3 to 6 membered carbocyclic or heterocyclic ring containing a heteroatom selected from sulfur, oxygen or NR⁰⁰⁰, wherein R⁰⁰⁰ is hydrogen or C₁₋₄alkyl, where the carbocyclic or heterocyclic ring is optionally substituted with halo or C₁₋₄alkyl,
R⁵⁰ is hydrogen, optionally substituted C₁₋₄alkyl, optionally substituted C₃₋₆cycloalkyl, optionally substituted aryl or optionally substituted heteroaryl containing a heteroatom selected from sulphur, oxygen or NR⁰⁰⁰, wherein R⁰⁰⁰ is hydrogen or C₁₋₆alkyl,
L is sulfur or oxygen, and
n is 0, 1 or 2, and
salts and N-oxides of the compounds of the formula 1.

2. Compounds according to claim 1, wherein Q² is hydrogen, Q¹ and Q³ are as defined in claim 1.

3. Compounds according to claim 1 wherein Q¹ is halogen, aryl or heteroaryl, Q² is hydrogen and Q³ is as defined in claim 1.

4. Compounds according to claim 1 wherein Q¹ is aryl, Q² is hydrogen and Q³ is as defined in claim 1.

5. Compounds according to claim 1, wherein Q¹ is heteroaryl, Q² is hydrogen and Q³ is as defined in claim 1.

6. Compounds according to claim 1, wherein Q¹ and Q³ are halogen and Q² is hydrogen.

7. Compounds according to claim 1 wherein, Q¹ is aryl or heteroaryl, Q² is hydrogen and Q³ is halogen.

8. Compounds according to claim 1, wherein Q¹ and Q² are hydrogen and Q³ is halogen or optionally substituted alkyl.

9. Compounds according to claim 1, wherein Q¹ is halogen, Q² is hydrogen and Q³ is optionally substituted alkyl.

10. Compounds according to claim 1, wherein Q¹ and Q² are halogen and Q³ is optionally substituted alkyl.

11. Compounds according to claim 1 wherein Q¹ is bromo.

12. Compounds according to claim 1 wherein Q¹ is iodo.

13. Compounds according to claim 1 wherein Q¹ is chloro.

14. Compounds according to claim 1 wherein Q¹ is fluoro.

15. Compounds according to claim 1 wherein Q³ is halogen.

16. Compounds according to claim 1 wherein Q³ is fluorine.

17. Compounds according to claim 1, wherein Q¹ is bromo, Q² is hydrogen and Q³ is fluoro.

18. Compounds according to claim 1, wherein Q¹ is bromo, Q² is hydrogen and Q³ is chloro.

19. Compounds according to claim 1, wherein Q¹ is iodo, Q² is hydrogen and Q³ is fluoro.

20. Compounds according to claim 1, wherein Q¹ is iodo, Q² is hydrogen and Q³ is chloro.

21. Compounds according to claim 1, wherein Q¹ is hydrogen, halogen, aryl or heteroaryl.

22. Compounds according to claim 1, wherein R¹ is C₁₋₄ alkyl or halo(C₁₋₄)alkyl.

23. Compounds according to claim 1, wherein R¹ is methyl.

24. Compounds according to claim 1, wherein R¹ is ethyl.

25. Compounds according to claim 1, wherein R² is hydrogen or methyl.

26. Compounds according to claim 1, wherein R² is hydrogen.

27. Compounds according to claim 1, wherein Q¹, Q² and Q³ are halogen.

28. Compounds according to claim 1, wherein Q¹ is halogen, Q² is C₁₋₄alkyl and Q³ is halogen.

29. Compounds according to claim 28, wherein Q¹ is halogen, Q² is C₁₋₄alkyl and Q³ is chloro.

30. Compounds according to claim 28 wherein Q¹ is halogen, Q² is C₁₋₄alkyl and Q³ is fluoro.

31. Compounds according to claim 1, wherein Q¹ is halogen, Q² is methyl and Q³ is halogen.

32. Compounds according to claim 31, wherein Q¹ is halogen, Q² is methyl and Q³ is chloro.

33. Compounds according to claim 31, wherein Q¹ is halogen, Q² is methyl and Q³ is fluoro.

34. Compounds according to claim 31, wherein Q¹ is bromo, Q² is methyl and Q³ is chloro.

35. Compounds according to claim 31, wherein Q¹ is bromo, Q² is methyl and Q³ is fluoro.

36. Compounds according to claim 31, wherein Q¹ is iodo, Q² is methyl and Q³ is chloro.

37. Compounds according to claim 31, wherein Q¹ is iodo, Q² is methyl and Q³ is fluoro.

38. Compounds according to claim 1, wherein Q¹ is halogen and Q² and Q³ are C₁₋₄alkyl.

39. Compounds according to claim 1, wherein R³ is tert-butyl, 1-halo-2-methylprop-2-yl, 1,1-dihalo-2-methylprop-2-yl, 1,1,1-trihalo-2-methylprop-2-yl, 1-alkoxy-2-methylprop-2-yl, 1-alkenyloxy-2-methylprop-2-yl, 1-alkynyloxy-2-methylprop-2-yl, 1-cyano-2-methyl-prop-2-yl, 1-alkoxyalkoxy-2-methyl-prop-2-yl, 1-halo-3-methylbut-3-yl, 1,1-dihalo-3-methylbut-3-yl, 1,1,1-trihalo-3-methylbut-3-yl, 1-alkoxy-3-methylbut-3-yl, 1-alkenyloxy-3-methylbut-3-yl, 1-alkynyloxy-3-methylbut-3-yl, 1-cyano-3-methylbut-3-yl, 2-cyanoprop-2-yl, 2-methoxycarbonylprop-2-yl or 2-methylaminocarbonylprop-2-yl, 1-alkylthio-2-methylprop-2-yl, 1-alkylsulphinyl-2-methylprop-2-yl, 1-alkylsulphonyl-2-methylprop-2-yl, 2-cyano-1-alkoxyprop-2-yl, 2-methyl-1-[(E and/or Z)-hydroxyimino]-prop-2-yl, 2-methyl-1-[(E and/or Z)-alkoxyimino]-prop-2-yl, 2-methyl-1-[(E and/or Z)-aryloxyimino]-prop-2-yl, 2-methyl-1-[(E and/or Z)-heteroaryloxyimino]-prop-2-yl, 1-alkoxy-prop-2-yl, 1-halo-prop-2-yl, 3-methyl-but-1-yn-3-yl, 1-alkyl-3-methyl-but-1-yn-3-yl, 4-methyl-pent-2-yn-4-yl, 1-hydroxy-4-methyl-pent-2-yn-4-yl, 1-alkoxy-4-methyl-pent-2-yn-4-yl, 1-alkoxyalkoxy-4-methyl-pent-2-yn-4-yl, 1-alkoxyalkoxyalkyl-4-methyl-pent-2-yn-4-yl, 1-cyanoalkyl-3-methylbut-3-yl, 1-haloalkyl-3-methylbut-3-yl.

40. Compounds according to claim 39, wherein R³ is tert-butyl, 1-halo-2-methylprop-2-yl, 1-fluoro-2-methylprop-2-yl, 1-methoxy-2-methylprop-2-yl, 1-ethoxy-2-methylprop-2-yl, 1-allyloxy-2-methylprop-2-yl, 1-(prop-2-ynyloxy)-2-methylprop-2-yl, 2-cyano-1-ethoxy-prop-2-yl, 2-cyano-1-methoxyprop-2-yl, 1-halo-3-methylbut-3-yl, 1-fluoro-3-methylbut-3-yl, 3-methylbut-1-yn-3-yl, 4-methylpent-2-yn-4-yl, 5-methyl-hex-3-yn-5-yl, 1-methoxy-4-methyl-pent-2-yn-4-yl, 1-allyloxy-4-methyl-pent-2-yn-4-yl, 1-propargyloxy-4-methyl-pent-2-yn-4-yl, 1-ethoxy-4-methyl-pent-2-yn-4-yl.

41. Compounds according to claim 1, wherein R⁴ is C₁₋₆alkyl optionally substituted with C₁₋₄alkoxy-(C₁₋₄)alkoxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with benzyloxy(C₁₋₄)alkyl, wherein the alkyl group is optionally substituted with halo, mono- or di-(C₁₋₆)alkylamino or tri(C₁₋₄)alkylsilyl, or R⁴ is C₁₋₆ alkyl optionally substituted with C₂₋₆ alkenyloxy or —S(O)ₓ(C₁₋₆)alkyl, wherein x is 0, 1 or 2 and the alkyl group is optionally substituted with halo, mono- or di-(C₁₋₆)alkylamino, —NH(C₁₋₄)alkyl=NOR, wherein R is hydrogen or C₁₋₄alkyl, or wherein the alkyl group is optionally substituted with tri(C₁₋₄)alkylsilyl, or R⁴ is —CR^{uu}═NR^{vv}, wherein R^{uu} is hydrogen or C₁₋₆ alkyl and R^{vv} is hydroxy or optionally substituted C₁₋₆alkoxy.

42. Compounds according to claim 1, wherein the optionally substituted aryl and optionally substituted heteroaryl rings or moieties of the R₅ values are optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)alkenyloxy, C₂₋₆ alkynyloxy, halo(C₂₋₆)alkynyloxy, aryl, aryloxy, aryl(C₁₋₆)alkyl, aryl(C₁₋₆)alkoxy, heteroaryl, heteroaryloxy, heteroaryl(C₁₋₆)alkyl, heteroaryl(C₁₋₆)alkoxy, —SF₅, —S(O)_{g}(C₁₋₄)alkyl wherein g is 0, 1 or 2 and the alkyl is optionally substituted with halo, or R⁵ is optionally substituted with —OSO₂(C₁₋₄)alkyl, wherein the alkyl group is optionally substituted with halo, or R⁵ is optionally substituted with —CONR^{g}R^{h}, —COR^{g}, —CO₂R^{g}, —R^{g}═NR^{h}, —NR^{g}R^{h}, —NR^{g}COR^{h}, —NR^{g}CO₂R^{h}, —SO₂NR^{g}R^{h} or —NR^{g}SO₂Rⁱ, wherein Rⁱ is C₁₋₆ alkyl optionally substituted with halogen and R^{g} and R^{h}, independently of each other, are hydrogen or C₁₋₆ alkyl optionally substituted with halogen, or, in the case of —CONR^{g}R^{h} or —SO₂NR^{g}R^{h}, R^{g}R^{h} may join to form a 5- or 6-membered carbocyclic or heterocyclic ring containing a heteroatom selected from sulphur, oxygen or NR⁰, wherein R⁰ is hydrogen or optionally substituted C₁₋₆alkyl.

43. Compounds according to claim 1, wherein the optionally substituted aryl, optionally substituted heteroaryl or optionally substituted 5- to 8-membered ring R⁴ is optionally substituted with halogen, cyano, nitro, azido, C₁₋₆ alkyl, halo(C₁₋₆)alkyl, C₃₋₆ cycloalkyl, C₃₋₆ cycloalkyl(C₁₋₄)alkyl, C₂₋₆ alkenyl, halo(C₂₋₆)alkenyl, C₂₋₆ alkynyl, halo(C₂₋₆)alkynyl, C₁₋₆ alkoxy, halo(C₁₋₆)alkoxy, C₂₋₆ alkenyloxy, halo(C₂₋₆)alkenyloxy, C₂₋₆ alkynyloxy, halo(C₂₋₆)alkynyloxy, —SF₅, —S(O)ₓ(C₁₋₆)alkyl, wherein x is 0, 1 or 2 and the alkyl group is optionally substituted with halo, or R⁴ is optionally substituted with —OSO₂(C₁₋₄)alkyl, wherein the alkyl group is optionally substituted with halogen, —CONR^{x}R^{y}, —CON(OR^{x})R^{y}, —COR^{x}, —CO₂R^{x}, —CR^{x}═NR^{y}, —NR^{x}R^{y}, —NR^{x}COR^{y}, —NR^{x}CO₂R^{y}, —SO₂NR^{x}R^{y} or —NR^{x}SO₂R^{z}, wherein R^{z} is C₁₋₈ alkyl optionally substituted with halogen and R^{x} and R^{y}, independently of each other, are hydrogen or C₁₋₆ alkyl optionally substituted with halogen.

44. Compounds according to claim 1, wherein R⁵⁰ is C₁₋₄alkyl optionally substituted with halogen, hydroxy, C₁₋₆alkoxy, C₂₋₆ alkenyl, C₂₋₆ alkynyl, C₁₋₄ alkoxy(C₁₋₄)alkoxy, cyano, C₁₋₄ alkylcarbonyloxy, aminocarbonyloxy, mono- or di(C₁₋₄)alkylamino-carbonyloxy, S(O)ₚ(C₁₋₆)-alkyl, wherein p is 0, 1 or 2, triazolyl, pyrazolyl, imidazolyl, tri(C₁₋₄)-alkylsilyloxy, optionally substituted phenoxy, optionally substituted thienyloxy, optionally substituted benzyloxy or optionally substituted thienylmethoxy.

45. Compounds according to claim 1, wherein R⁵⁰ is C₃₋₆cycloalkyl optionally substituted with halogen, hydroxy, C₁₋₆alkoxy, C₁₋₄ alkoxy(C₁₋₄)alkoxy, cyano, C₁₋₄alkylcarbonyloxy, aminocarbonyloxy, mono- or di(C₁₋₄)alkylamino-carbonyloxy, S(O)ₚ(C₁₋₆)-alkyl, wherein p is 0, 1 or 2, triazolyl, pyrazolyl, imidazolyl, tri(C₁₋₄)-alkylsilyloxy, optionally substituted phenoxy, optionally substituted thienyloxy, optionally substituted benzyloxy or optionally substituted thienylmethoxy,

46. Compounds according to claim 1, wherein the optionally substituted aryl or the optionally substituted heteroaryl R⁵⁰ is optionally substituted with halogen, hydroxy, mercapto, C₁₋₄ alkyl, C₂₋₄ alkenyl, C₂₋₄ alkynyl, C₁₋₄ alkoxy, C₂₋₄ alkenyloxy, C₂₋₄ alkynyloxy, halo(C₁₋₄)alkyl, halo(C₁₋₄)alkoxy, C₁₋₄ alkylthio, halo(C₁₋₄)-alkylthio, hydroxy(C₁₋₄)alkyl, C₁₋₄ alkoxy(C₁₋₄)alkyl, C₃₋₆cycloalkyl, C₃₋₆ cycloalkyl-(C₁₋₄)alkyl, phenoxy, benzyloxy, benzoyloxy, cyano, isocyano, thiocyanato, isothiocyanato, nitro, NR^{p}R^{q}, NHCOR^{p}, —NHCONR^{p}R^{q}, CONR^{p}R^{q}, —SO₂R^{o}, OSO₂R^{o}, COR^{p}, CR^{p}═NR^{q} or N═CR^{p}R^{q}, wherein R^{o} is C₁₋₄ alkyl, halo(C₁₋₄)alkyl, C₁₋₄alkoxy, halo(C₁₋₄)alkoxy, C₁₋₄alkylthio, C₃₋₆ cycloalkyl, C₃₋₆cycloalkyl(C₁₋₄)alkyl, phenyl or benzyl, the phenyl and benzyl groups being optionally substituted with halogen, C₁₋₄ alkyl or C₁₋₄ alkoxy, and R^{p} and R^{q} are independently hydrogen, C₁₋₄alkyl, halo(C₁₋₄)alkyl, C₁₋₄alkoxy, halo(C₁₋₄)alkoxy, C₁₋₄alkylthio, C₃₋₆ cycloalkyl, C₃₋₆cycloalkyl(C₁₋₄)alkyl, phenyl or benzyl, the phenyl and benzyl groups being optionally substituted with halogen, C₁₋₄alkyl or C₁₋₄alkoxy.

47. Compounds according to claim 1, wherein L is oxygen.

48. Compounds according to claim 1, wherein n is 0.

49. A process for preparing a compound of the formula I according to claim 1 as herein described.

50. A fungicidal composition comprising a fungicidally effective amount of a compound according to claim 1 and a suitable carrier or diluent therefor.

51. A method of combating or controlling phytopathogenic fungi which comprises applying a fungicidally effective amount of a compound according to claim 1 to a plant, to a seed of a plant, to the locus of the plant or seed or to soil or any other plant growth medium.
